(19) United States
(12) Patent Application Publication
(10) Pub. No.: US 2005/0123993 A1
(43) Pub. Date: Jun. 9, 2005

(54) Methods of determining ligand residue binding affinity

(76) Inventors: Stephan Brunner, Preverenges (CH); David Mosenkis, Philadelphia, PA (US); Frank Hollinger, Wayne, PA (US); William Chiang, Pennington, NJ (US)

Correspondence Address: STERNE, KESSLER, GOLDSTEIN & FOX PLLC, 1100 NEW YORK AVENUE, N.W., WASHINGTON, DC, 20005, US

(21) Appl. No.: 10/730,267

(22) Filed: Dec. 9, 2003

(51) Int. Cl. G01N 33/53; G06F 19/00; G01N 33/48; G01N 33/50

(52) U.S. Cl. 435/7.1; 702/19

(57) ABSTRACT

Methods and systems for determining the affinity between polypeptide amino acid residues and one or more molecular fragments, and for using the affinity values to aid in drug design including a computer simulation which calculates the interaction energy between a polypeptide and at least one molecular fragment. An affinity value is then assigned to at least one fragment and residue pair if the fragment is in the vicinity of the residue. Affinity values are used to rank fragments, build ligands and determine binding sites.

### BACKGROUND OF THE INVENTION

1. Field of the Invention

The present invention relates to computer-implemented methods and systems of determining the affinity between polypeptide amino acid residues and one or more molecular fragments. The invention further provides methods and systems of using the affinity values to aid in drug design.

2. Related Art

The action of a particular drug is believed to be due to the interaction of that drug with a particular molecular target, such as a protein, nucleic acid, or other molecule found in the biological system. Typical protein drug targets include enzymes and receptors. Thomas, G., “Medicinal Chemistry—An Introduction” (John Wiley & Sons, Ltd., New York, 2001). The binding of the drug to the active or other sites (allosteric sites) of an enzyme usually has the effect of preventing the normal operation of that enzyme. Similarly, drugs act on receptors by binding to or near to a specific receptor that may either activate the receptor or prevent the binding of the receptor's normal substrate to that receptor. Ultimately, both of these actions can result in a physiological response that may have a therapeutic effect. The drug's effectiveness will depend on the stability of the drug-enzyme or drug-receptor complex and the number of sites occupied by the drug. Other targets for drug action include nucleic acids and other naturally occurring molecules. Id.

To rationally develop a drug lead, therefore, it is desirable to know the binding site on the target molecule (e.g., enzyme, receptor or nucleic acid). One approach to determining protein binding sites is protein mapping, where molecular probes, such as small organic molecules or functional groups are placed around the protein surface to determine the most favorable binding positions (Dennis et al., PNAS 99:4290-4295 (2002)). Experimental approaches to protein mapping include x-ray crystallography and NMR methods. Id. Both of these approaches have shown that probes, even those generally unrelated to any natural substrate of the protein, bind to only a limited number of positions on the protein. Generally, a pocket of the active site tends to form a consensus site that binds many ligands, regardless of their sizes and polarities. Id.

Because of the experimental difficulties associated with co-crystallizing proteins and probes, or the use of NMR to determine binding sites, a number of methods have been developed to perform mapping computationally rather than experimentally (e.g, the drug design program GRID (Goodford, P. J., J. Med. Chem. 28:849-875 (1985) and the multiple copy simultaneous search (MCSS) strategy (Miranker, A. & Karplus, M., Proteins Struct. Funct. Genet. 11:29-34 (1991)).

The major problem with these approaches, however, is that they result in many energy minima along the surface of the protein, making it difficult to determine which of the minima is actually relevant (Dennis et al., PNAS 99:4290-4295 (2002)).

In one approach, a mapping algorithm was developed that uses an empirical free energy function to determine the active sites of egg white lysozyme and thermolysin. Id. This study reported that the sites identified by computational mapping agreed with those identified experimentally.

The MCSS tool, discussed above, uses a fragment-based computational approach to identify binding sites and, as discussed above, is essentially based on an energy minimization approach, providing fragment states corresponding to various local minima of the fragment-protein interaction potential energy field.

Improved computational methods are necessary to provide accurate, quantitative estimates of the free energy of binding of molecular fragments to potential binding sites so that ligands can be designed for these sites. Such a computational method should yield values which can be used to determine the affinity of particular fragment-residue pairs, so that the results of particular simulations can be compared, the degree of convergence of a particular simulation can be determined and binding sites and key fragments can be identified.

### SUMMARY OF THE INVENTION

Recognizing the tremendous need for accurate determinations of the interaction energies between fragments and the amino acid residues of polypeptide molecules, the present inventors have developed methods and systems of determining the affinity values of fragment-residue pairs.

Accordingly, the present invention provides methods and systems of determining the affinity between polypeptide amino acid residues and one or more molecular fragments. The invention includes conducting a computer simulation of (i) a polypeptide and (ii) at least one molecular fragment, wherein at least one interaction energy is calculated between the polypeptide and at least one molecular fragment, wherein each calculated interaction energy is associated with a position of said at least one molecular fragment; and assigning an affinity value to at least one fragment and residue pair when said fragment is in the vicinity of the residue, wherein said affinity value is a measure of the free energy of interaction between the polypeptide and the fragment; wherein the above calculations are conducted for each molecular fragment present in the computer simulation.

The present invention further provides methods and systems of using the affinity values of the present invention to, for example, determine the degree of convergence of a particular simulation, compare the results of multiple computer-implemented simulations, identify protein binding sites, and help determine the key fragments to use in constructing ligands for a given polypeptide.

### DETAILED DESCRIPTION OF THE INVENTION

Terms are used herein as generally used in the art, unless otherwise defined herein.

In one aspect, the present invention provides methods and systems of determining the affinity between polypeptide amino acid residues and one or more molecular fragments. In one embodiment, the present invention includes conducting a computer simulation of (i) a polypeptide, and (ii) at least one molecular fragment, wherein at least one interaction energy is calculated between said polypeptide and said at least one molecular fragment, wherein each calculated interaction energy is associated with a position of said at least one molecular fragment; and assigning an affinity value to at least one fragment and residue pair when said fragment is in the vicinity of the residue, wherein said affinity value is a measure of the free energy of interaction between the polypeptide and the fragment; wherein the interaction energy and the affinity value is determined for each molecular fragment present in the computer simulation.

As used herein, the term “polypeptide” encompasses a molecule comprised of amino acid molecules linked by peptide bonds, and includes all such molecules, regardless of the number of amino acids in the molecule. The term polypeptide, as used herein, also includes molecules which include other moieties in addition to amino acids, such as glycosylated polypeptides, e.g., antibodies. The term polypeptide, as used herein, also includes protein molecules which consist of more than one chain of amino acids linked by peptide bonds; the multiple chains may be covalently bonded to each other by means of disulfide sidechain bonds.

“Fragments,” as the term is used herein, includes molecules or molecular fragments (e.g., radicals) that can be used to model one or more interaction with a macromolecule, such as the interactions of carbonyls, hydroxyls, amides, hydrocarbons, and the like. Examples of useful fragments include:

| Name | Structure |
|---|---|
| Acetone | CH₃(C═O)CH₃ |
| Aldehyde | H(C═O)—CH₃ |
| Amide | H(C═O)NH₂ |
| Ammonia | NH₃ |
| Benzene | 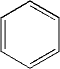 |
| Carboxylic Acid | CH₃COOH |
| 1,4-Diazine | 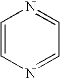 |
| Ester | CH₃—O—(C═O)—CH₃ |
| Ether | CH₃—O—CH₃ |
| Formaldehyde | H₂C═O |
| Furan | 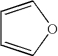 |
| Imidazole | 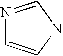 |
| Methane | CH₄ |
| Methanol | CH₃OH |
| Phospho-Acid | 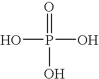 |
| Pyridine | 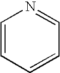 |
| Pyrimidine | 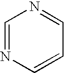 |
| Pyrrole | 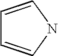 |
| Thiol | CH₃SH |
| Thiophene | 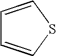 |

Preferably, the fragments selected are representative of chemical features that have proven useful in the design of pharmaceuticals or other bioactive chemicals. Additional fragments will be readily apparent to one skilled in the art.

In one aspect of the methods of the present invention, a computer simulation of a polypeptide and at least one molecular fragment is conducted, wherein at least one interaction energy is calculated between the polypeptide and the at least one molecular fragment, wherein each calculated interaction energy is associated with a position of the molecular fragment. For example, in one aspect of the present invention, the calculation of interaction energy between the polypeptide and each molecular fragment, uses a Monte Carlo method to explore the protein/fragment confirmation space.

In one embodiment, the three dimensional structure of a target protein, usually obtainable experimentally from x-ray crystallography, is known and the basic interactions between the protein and the small fragments (e.g., average molecular weight of approximately 150) are computed. This computation can be carried out by Monte Carlo (MC) modeling and analysis (usually implemented in software) for a large collection of organic fragments with diverse physico-chemical properties. The number of fragments can be in the hundreds to thousands. For this purpose, Locus Pharmaceuticals, Inc., Blue Bell, Pa., developed the Locus Monte Carlo (LMC) code. For each rigid fragment instance, a set of attributes are saved, including:
(x,y,z), q=(q₁, q₂, q₃, q₄), fragment-protein energy,
where (x,y,z) are the coordinates of the fragment's center of mass, and q is the quaternion characterizing its orientation.

This MC data for the different fragments is analyzed for identifying potential binding sites using the methods of the present invention. These tools are based on the postulate that a binding site must be a high affinity region for a diverse collection of fragments. In one aspect, experimental binding site data (e.g., co-crystal X-ray data and residue mutational analysis), if available, is used to determine the final site within which the leads are designed.

In another aspect, the actual thermodynamic fragment distributions around the protein, i.e., distributions consistent with thermal fluctuations at physiological temperatures are calculated. Information on the thermodynamic distribution is essential for computing free energies of binding, which is the basic biologically relevant quantity for quantifying the binding affinity of a ligand. The MCSS approach, by contrast, is essentially based on an energy minimization approach, providing fragment states corresponding to various local minima of the fragment-protein interaction potential energy field. Such a procedure is computationally more expeditious than computing the actual physical distributions, but is unable to provide information on entropic effects, essential for free energy estimates.

For computing the thermodynamic distributions, MC simulation packages often make use of a Metropolis Monte Carlo approach (Metropolis, N., et al., J. Chem. Physics 21:1087-1092 (1953)) for sampling from a grand-canonical ensemble of states (Adams, D. J., Molecular Physics 29:307-311 (1975); Mezei, M., Molecular Physics 61:565-582 (1987)). In addition to exchanging just energy with a surrounding thermal bath, as in the case of a canonical ensemble, the system described by a grand-canonical ensemble may exchange particles or fragments as well. The energy cost associated with inserting/deleting a fragment from the system is controlled by its chemical potential. By varying this chemical potential, so-called simulated annealing of the chemical potential, one may vary the average number of fragments in the simulation system. It can be shown that measuring the values of the chemical potential at which fragments leave various sites on the protein provides an estimate of the free energy of binding for the different binding modes over the protein surface.

The practicality of the simulated annealing procedure for estimating binding affinities was demonstrated by Guarnieri and Mezei for differentiating hydration propensities of different DNA grooves (Guarnieri, F. and Mezei, M., J. Am. Chem. Soc. 118:8493-8494 (1996)). These results were obtained with the Metropolis Monte Carlo (MMC) code developed by the group of Mezei, Mount Sinai School of Medicine, N.Y. For these simulations, the system was composed of a molecule fraction of DNA surrounded by a varying number of interacting water molecules. In one embodiment, the LMC algorithm carries out a similar calculation for all fragments with respect to the target protein.

Accordingly, in an embodiment of the present invention, a macromolecule is analyzed for potential binding sites. For example, this analysis can be accomplished by (1) positioning an instance of a computer representation of a molecule or molecular fragment at a plurality of sampling sites of the macromolecule; (2) selecting a value of B, wherein B=μ′/kT+ln <N>, where μ′ is the excess chemical potential, k is Boltzmann's constant, T is the absolute temperature, and <N> is the mean number of molecules of the molecule or molecular fragment; (3)repositioning the instances of the molecule or molecular fragment; (4)accepting or rejecting each instance of the repositioned molecule or molecular fragment based on the Metropolis sampling criteria using the computed binding energy compared to the selected value of B; (5) repeating steps (1) through (4) at a lesser value of B; and outputting a list of unrejected instances of the molecule or molecular fragment; wherein the molecule or molecular fragment is an organic fragment.

In an embodiment, the sampling sites comprise an unbiased sampling of sites of the macromolecule.

In another aspect of the invention, a Monte Carlo based computer simulation is conducted which excludes the fragment-fragment interactions. It has been acknowledged that considering fragment-fragment interactions may be detrimental to the interpretation of the simulation results for all fragments but water. Indeed, due to the high dilution of the solute molecules in actual biochemical relevant conditions, considering interactions between non-water fragments may not be realistic. Furthermore, the drug leads assembled by LCD usually are composed of only one fragment of each type. Fragment-fragment interactions in a simulation may thus lead to detrimental correlation effects.

Therefore, in an embodiment, the interaction between a given fragment and a protein is analyzed by sampling the fragment states from a thermodynamically relevant Grand-Canonical distribution. The underlying sampling algorithm is a weighted Metropolis Monte Carlo approach, described herein as weighted Grand-Canonical Metropolis Monte Carlo (WGCMMC) sampling. The weighting procedure is implemented by subdividing space with an orthogonal, equidistant grid. Each grid cell x is assigned a local, numerical chemical potential field value Bₙᵤₘ,(x), which is adapted iteratively to ensure an approximately uniform numerical sampling of fragment states around the protein. Bₙᵤₘ is related to the thermodynamic cost of inserting or removing a fragment, and its local value defines the weight for each sampled fragment state.

Once the Bₙᵤₘ field has sufficiently converged, and the Markov chain associated to the MC sampling has equilibrated, the Markov chain can be sampled periodically at successive decorrelated states. Positions, orientations, potential energies and statistical weights for all fragment states are saved. Binding modes are then identified and corresponding binding free energies estimated.

This approach makes use of a Grand-Canonical Metropolis Monte Carlo algorithm for sampling fragments around the target protein. This sampling data can then be directly used for estimating the free energy of binding for different binding modes of the fragment on the protein surface. This approach distinguishes itself from the MMC process, in that it removes fragment-fragment interactions.

It turns out that the standard Monte Carlo approach has difficulty in handling simulations where fragment-fragment interactions are removed. Indeed, the absence of fragment-fragment interactions can lead to a broad range of fragment densities between the high and low affinity binding sites on the protein. This results from the possible overlap of fragments. The standard Metropolis Monte Carlo scheme used in conventional approaches has trouble resolving this dynamical range in densities. This problem is overcome with a weighted Monte Carlo scheme.

Therefore, in an embodiment, a linear Monte Carlo approach (i.e., without fragment-fragment interactions) is used to calculate the protein-fragment interaction energies. This scheme can be described as “linear” in reference to the linear properties of the Liousville equation in the absence of fragment-fragment interactions. Liousville's equation describes the time evolution of the system away from equilibrium.

First, the derivation of the grand canonical distribution for WGCMMC is presented below.

The potential energy of the system composed of N fragments is denoted U(Γ, N). In general, U includes both contributions from fragment-protein and fragment-fragment interactions. The configuration of the system is characterized by
Γ=(Y₁,Y₂, . . . ,Y_{N}), (1)
where Yᵢ=(xᵢ,Ωᵢ) stands for the position xᵢ and orientation Ωᵢ of fragment i.

In the grand canonical ensemble, the probability that the system has N fragments in configuration Γ is given by

$$f ( Γ , N ) = \frac{1}{Q} \frac{1}{{V}^{N} {σ}^{N}} \frac{1}{N !} exp [ BN - β U ( Γ , N ) ] , \qquad ( 2 )$$

with the normalization factor given by the grand partition function

$$Q = {\sum }_{N = 0}^{∞} \frac{1}{N !} exp ( BN ) \int \frac{ⅆ {Y}^{N}}{{V}^{N} {σ}^{N}} exp [ - β U ( Γ , N ) ] . \qquad ( 3 )$$

Here V is the volume of the system, φ is the volume of orientational space, and B is related to the excess chemical potential μ^{ex}, i.e. the energy cost in units of β⁻¹=KT for a particle to leave the system:
B=βμ^{ex}+log\overline{N}, (4)
where N is the average number of fragments in the system.

Assuming no fragment-fragment interactions, the potential energy U of the system becomes:

$$U ( Γ , N ) = {\sum }_{i = 1}^{N} E ( {Y}_{i} ) , \qquad ( 5 )$$

where E(Yᵢ) is the energy of interaction of the ith fragment with the protein.

The Grand Partition Function can then be written as

$$\begin{matrix}Q = {\sum }_{N = 0}^{∞} \frac{1}{N !} {( exp ( B ) \int \frac{ⅆ Y}{V σ} exp [ - β E ( Y ) ] )}^{N} = exp Z , \\ with & ( 6 ) \\ Z = exp ( B ) \int \frac{ⅆ Y}{V σ} exp [ - β E ( Y ) ] . & ( 7 )\end{matrix}$$

The probability P(N) for having N fragments in the system is then given by

$$P ( N ) = \int ⅆ {Y}^{N} f ( Γ , N ) = exp ( - Z ) \frac{{Z}^{N}}{N !} . \qquad ( 8 )$$

As expected, this is simply the Poisson distribution with parameter Z. In particular, the average number of fragments in the system is given by

$$〈 N 〉 = {\sum }_{N = 1}^{∞} NP ( N ) = Z , \qquad ( 9 )$$

which thus scales exponentially with B.

In fact, more generally, the probability P(n,ΔV) of finding n fragments in any given subvolume ΔV of configuration space is given by a Poisson distribution:

$$\begin{matrix}\begin{matrix}P ( n , Δ V ) ∼ {\sum }_{N = n}^{∞} \frac{N !}{( N - n ) ! n !} {\int }_{Δ V} ⅆ {Y}_{1} … ⅆ {Y}_{n} \\ \int ⅆ {Y}_{n + 1} … ⅆ {Y}_{N} f ( Γ , N ) \\ = \frac{1}{n !} {( exp ( B ) \int \frac{ⅆ Y}{V σ} exp [ - β E ( Y ) ] )}^{n} \\ \frac{1}{Q} {\sum }_{N = n}^{∞} \frac{{Z}^{N - n}}{( N - n ) !} \\ = {z}^{n} / n ! ,\end{matrix} \\ with & ( 10 ) \\ z = exp ( B ) {\int }_{Δ V} \frac{ⅆ Y}{V σ} exp [ - β E ( Y ) ] . & ( 11 )\end{matrix}$$

Finally, the single fragment density is given by

$$\begin{matrix}{f}_{gc} ( Y ) = {\sum }_{N = 1}^{∞} N \int ⅆ {Y}_{2} … \int ⅆ {Y}_{N} f ( Γ = ( Y , {Y}_{2} , … , {Y}_{N} ) , N ) \\ = exp ( - Z ) \frac{1}{V σ} exp [ B - β E ( Y ) ] {\sum }_{N = 1}^{∞} \frac{1}{( N - 1 ) !} {Z}^{( N - 1 )} \\ = \frac{1}{V σ} exp [ B - β E ( Y ) ] ,\end{matrix} \qquad ( 12 )$$

which again scales exponentially with respect to B.

Equation (12) for the single fragment density shows the large dynamical range that may result from the exponential dependence of this quantity with respect to the single fragment-protein potential energy E(Y). This dependence results from the possible overlap of the non-interacting fragments. This was not an issue in the presence of fragment-fragment interactions, as an upper bound to the fragment density was then set by the tightest possible packing of the molecules.

The underlying method developed for WGCMMC to enable the accurate resolution of the above-mentioned dynamical range in densities is presented here.

For numerical purposes, instead of considering a constant B value, one may consider a field Bₙᵤₘ(Y) in the single particle configuration space Y. This field represents the energy cost for a particle to leave the system specifically from position Y. In this case, the density of states in the grand canonical ensemble (2) is given by

$${f}_{num} ( Γ , N ) = \frac{1}{{Q}_{num}} \frac{1}{{V}^{N} {σ}^{N}} \frac{1}{N !} exp [ {\sum }_{i = 1}^{N} {B}_{num} ( {Y}_{i} ) - β U ( Γ , N ) ] , \qquad ( 14 )$$

with the normalization factor (grand partition function) now given by

$${Q}_{num} = {\sum }_{N = 0}^{∞} \frac{1}{N !} \int \frac{ⅆ {Y}^{N}}{{V}^{N} {σ}^{N}} exp [ {\sum }_{i = 1}^{N} {B}_{num} ( {Y}_{i} ) - β U ( Γ , N ) ] . \qquad ( 15 )$$

A similar derivation as the one used for obtaining Eq. (12) leads to the corresponding single fragment density:

$${f}_{gc , num} ( Y ) = \frac{1}{V σ} exp [ {B}_{num} ( Y ) - β E ( Y ) ] . \qquad ( 16 )$$

Equation (16) shows that through the field Bₙᵤₘ(Y), the amplitude of the density in each position Y of the single particle configuration space can be calculated. Thus, by iteratively adapting Bₙᵤₘ(Y) during the convergence phase of the Metropolis MC simulation, one may obtain appropriate sampling in all regions of interest. This is achieved by taking
Bₙᵤₘ(Y)≅min (βE(Y)+const, Bₘₐₓ), (17)
leading to similar numerical densities of fragment instances in various regions of space. An upper bound Bₘₐₓ is set on Bₙᵤₘ to avoid unnecessary sampling in strongly unfavorable positions, i.e., essentially for configurations leading to steric clashes. In practice, the field Bₙᵤₘ(Y) is chosen to be independent of the fragment orientation, and to be piece-wise constant on a 3-D grid in x-space (translational-space).

Making use of the exponential dependence in B of the density, one can infer the physical fragment density f_{gc}(Y) at any B=B₀=constant value from the simulation results for a given numerical Bₙᵤₘ(Y) field. Assume that one has a sampling {Γᵢ=(Y₁, . . . ,Y_{N}_{ᵢ})}_{i=1, . . . ,n}_{ₛₙₐₚ} of nₛₙₐₚ snapshots from the numerical distribution f_{gc,num}(Γ,N). The average of any single fragment quantity A(Y) over the distribution f_{gc}(Y) is then given by

$$\begin{matrix}〈 A 〉 = \int ⅆ Y {f}_{gc} ( Y ) A ( Y ) = \int ⅆ Y {f}_{gc , num} ( Y ) \frac{{f}_{gc} ( Y )}{{f}_{gc , num} ( Y )} A ( Y ) \\ ≃ \frac{1}{{n}_{snap}} {\sum }_{i = 1}^{{n}_{snap}} {\sum }_{j = 1}^{{N}_{i}} {w}_{j} A ( {Y}_{j} ) ,\end{matrix} \qquad ( 18 )$$

where wⱼ is the weight assigned to the fragment state Yⱼ, and defined by

$${w}_{j} = \frac{{f}_{gc} ( {Y}_{j} )}{{f}_{gc , num} ( {Y}_{j} )} = exp ( {B}_{0} - {B}_{num} ( {Y}_{j} ) ) . \qquad ( 19 )$$

The scan over the B schedule carried out in an MMC-type annihilation simulation is thus replaced in a WGCMMC run by a simulation for a single Bₙᵤₘ(Y) field.

The following addresses how the WGCMMC data can be handled and analyzed.

The starting point for the data interpretation is the relation linking the WGCMMC data to the association constant Kₐ characterizing the binding of the considered fragment to a given region on the protein. As a reminder, this relation is rederived here.

The association constant Kₐ characterizes the equilibrium of the binding process
F+P⇄FP, (20)
and is defined by

$${K}_{a} = \frac{[ FP ]}{[ F ] [ P ]} , \qquad ( 21 )$$

where [P], [F], and [FP] are respectively the concentrations of protein alone, fragment alone, and of a particular protein-fragment complex (binding mode). The association constant is indeed the basic biologically relevant quantity.

In the case of the LMC system, consider a single protein in a volume V. For the sake of the following discussion, take V to be large, although for the actual MC simulation this need not be the case. The protein concentration is therefore given by [P]=1/V. Furthermore, note that n is the average number of fragments in the binding volume ΔV_{b} (in general a volume with limits both in translational and orientational space), and N is the average total number of fragments in the system, so that [F](N−n)/V and [FP]=n/V. The association constant can thus be written

$${K}_{a} = \frac{n / V}{( N - n ) / V1 / V} ≃ V \frac{n}{N} \qquad ( 22 )$$

having invoked the thermodynamic limit of large volume V, so that n<<N (N/V Π const, for V Π∞). The values n and N can be obtained from the fragment density (12):

$$\begin{matrix}n = {\int }_{Δ {V}_{b}} ⅆ Y {f}_{gc} ( Y ) = \frac{{e}^{B}}{V σ} {\int }_{Δ {V}_{b}} ⅆ Y exp [ - β E ( Y ) ] , & ( 23 ) \\ N = {\int }_{V} ⅆ Y {f}_{gc} ( Y ) = \frac{{e}^{B}}{V σ} {\int }_{V} ⅆ Y exp [ - β E ( Y ) ] ≃ {e}^{B} , & ( 24 )\end{matrix}$$

having again invoked the assumption of the high protein dilution, so that the total system volume V is much larger than the effective region of interaction between the fragment and the protein and thus one may consider E(Y)≅0 in deriving Eq. (24). The association constant now becomes:

$${K}_{a} = \frac{1}{σ} {\int }_{Δ {V}_{b}} ⅆ Y exp [ - β E ( Y ) ] . \qquad ( 25 )$$

On the basis of Eq. (25) one can also write the association constant in terms of the free energy of binding ΔA
Kₐ=V exp (−βΔA). (26)
where ΔA=A_{FP}−A_{F}, with A_{FP} and A_{F} the free energies of the fragment-protein complex and of the fragment alone respectively:

$$\begin{matrix}{A}_{FP} = - \frac{1}{β} log ( {\int }_{Δ {V}_{b}} ⅆ Y exp [ - β E ( Y ) ] ) , & ( 27 ) \\ {A}_{F} = - \frac{1}{β} log {\int }_{V} ⅆ Y = - \frac{1}{β} log ( V σ ) . & ( 28 )\end{matrix}$$

The critical value B_{c} that is associated to the binding volume ΔV_{b} can be defined as the value for which the average number of fragments in the binding site is 1. From Eq. (23) follows:

$$n ( {B}_{c} ) = 1 ⇔ {ⅇ}^{- {B}_{c}} = \frac{1}{{V}_{σ}} {\int }_{Δ {V}_{b}} ⅆ Y exp [ - β E ( Y ) ] , \qquad ( 29 )$$

and from (25), (26) and (29) one finally obtains:

$$Δ A = \frac{1}{β} {B}_{c} . \qquad ( 31 )$$

Thus, a low Bc value reflects a high affinity binding mode, and inversely a higher Bc value reflects a lower affinity mode.

The critical value B_{c} can be computed from the WGCMMC data using definition (29), as well as Eqs (18) and (19):

$$1 = n ( {B}_{c} ) = \frac{1}{{n}_{snap}} {\sum }_{i = 1}^{{n}_{snap}} {\sum }_{fragj ∈ Δ {V}_{b}} exp [ {B}_{c} - {B}_{num} ( {Y}_{j} ) ] \\ ⇔ {B}_{c} = - log [ \frac{1}{{n}_{snap}} {\sum }_{i = 1}^{{n}_{snap}} {\sum }_{frag j ∈ Δ {V}_{b}} {ⅇ}^{- {B}_{num} ( {Y}_{j} )} ] . \qquad ( 32 )$$

Equations (30), (31) and (32) provide the basic relations for how the WGCMMC data is to be interpreted.

Following the computer simulation which calculates the interaction energy between a polypeptide and at least one molecular fragment, an affinity value is assigned to at least one fragment and residue pair when the fragment is in the vicinity of the residue. A fragment is defined as being in the vicinity of a residue when at least one pair of fragment-residue atoms (i,j) is within a predetermined threshold distance, wherein said threshold distance is based on the sum of the Van der Waals radii of said fragment-residue atoms. In an embodiment of the present invention, the predetermined threshold distance is defined as follows:
rᵢⱼ<α(R_{VdW,i}+R_{VdW,j}) (32)
wherein rᵢⱼ is the distance between the two atoms, R_{VdW} is the Van der Waals radius and α is a numerical parameter. In an embodiment, α is between about 0.5 and about 2.0, and preferably is about 1.2. In one aspect the Van der Waals radius is about half the Lennard-Jones parameter from a molecular mechanics force-field. In an aspect of the present invention, the molecular mechanics force field is selected from the group consisting of AMBER, GROMOS, CHARMM, Xplor, Discover, MMFFF and Tripos. AMBER is a particularly preferred force field.

As discussed above, the affinity value that is assigned to any particular fragment-residue pair is a measure of the free energy of interaction between the polypeptide and fragment, thus, both enthalpic and entropic contributions are included.

In an aspect of the present invention, when a Monte Carlo based computer simulation is used which includes fragment-fragment interactions, and a simulated annealing of chemical potential is conducted, the affinity value comprises B-critical. B critical is defined as the minimum B value for which a particular fragment is persistently observed in the vicinity of a residue, wherein B=μ′/kT+ln <N>, where: ′ is the excess chemical potential, k is the Boltzmann's constant, T is the absolute temperature, and <N> is the mean number of molecules of the molecular fragment. In an embodiment, a particular type of fragment is persistently observed in the vicinity of a residue when the average number of fragments in the vicinity of the residue is between 0.8 and 1.0. In another aspect of the present invention, a particular type of fragment is persistently observed in the vicinity of a residue when the average number of fragments in the vicinity is greater than or equal to 0.9.

In another embodiment, with respect to linear Monte Carlo, B-critical is also used to determine the fragment/residue affinity. Accordingly, the binding affinity of a fragment for different regions on the protein surface can be estimated by assigning a critical B, to each fragment-residue pair. These B_{c} values are obtained from the WGCMMC data by applying relation (32), where the volume ΔV_{b} is approximated for each residue on the basis of a proximity criteria.

The volume defined on the basis of the proximity criteria may only be an estimate of a binding mode volume. The corresponding B_{c} values must therefore be used as estimates of free energy of binding. Nonetheless, comparing sets of B_{c} values for different fragments has proven valuable to help identify protein binding sites as follows: A binding site is identified as a set of residues with low B_{c} values (high affinity) for multiple fragments with diverse physico-chemical properties. This approach is based on the assumption that diverse interactions in a localized region are the necessary condition for ensuring the specificity of a binding site. Preferably, this numerical localization of the binding site is complemented by experimental binding information such as co-crystal X-ray data and mutational analysis.

Improved estimates for the binding mode volumes ΔV_{b}, compared to the above described residue-based proximity criteria, provide more accurate estimates of free energy of binding using Eq. (32). Such improved binding mode volume estimates are determined and represented by clustering sampled fragment states belonging to the same potential energy well. For this purpose the potential energies saved for the sampled fragment states are used.

In another aspect of the present invention, following the computer simulation of the polypeptide and at least one fragment and the assignment of affinity values to specific fragment/residue pairs, a binding analysis profile is outputted that comprises a matrix of affinity values for each fragment-residue pair.

In an embodiment of the present invention, numerous separate computer simulations are conducted on a particular polypeptide, wherein in each simulation a different fragment type interacts with the protein. For example, a simulation of polypeptide A is conducted with fragment X, wherein interaction energies are calculated, and affinity values assigned to fragment/residue pairs as described above. A computer simulation of polypeptide A is then conducted with fragment Y, wherein interaction energies are calculated, and affinity values assigned to fragment/residue pairs as described above, etc.

When separate simulations are conducted for a given polypeptide, a separate affinity value matrix can be generated for each fragment type. In this way the output can enable a ranking of the residues with respect to average fragment-binding ability for a given residue. A matrix of affinity values can be generated which is averaged over fragments types, and the polypeptide surface is coded according to average fragment binding affinity. For example, residues with highest fragment binding affinity values are a different color from the residues with the lowest affinity values. In this way, potential binding sites on the macromolecule can be identified. For example, the output can be visual, displaying the residues on the macromolecule's surface in different colors across the visible light spectrum from red (highest average residue-fragment affinity) to blue (lowest average residue-fragment affinity). In such an output, the potential binding sites with higher probabilities of being actual binding sites will appear as groups of residues colored red or closer to red in the spectrum.

The residue-fragment affinity can also be used to measure the degree of convergence of a simulation. For example, a matrix of B-critical values for each residue-molecular fragment pair can be created (an “affinity profile”). Convergence is considered to have occurred when the affinity profile remains constant (or stops changing) within a predetermined threshold range.

The residue-fragment affinity can also be used to measure the degree of difference between simulations. For example, the affinity profile of two or more simulations can be compared, and the absolute and statistical measures of their variance can be calculated.

The residue-fragment affinity can also be used to identify key fragments which can be used to design ligands (i.e., drug candidates). For one or more selected residues, molecular fragments can be ranked according to affinity value. For example, for a selected residue, the molecular fragments can be listed in ascending or descending order of residue-fragment affinity. Similarly, in an embodiment, the invention enables the display of a table of residues for each fragment that highlights the regions on the protein for which the fragment has the highest affinity.

The present invention is described in further detail in the following non-limiting examples.

### EXAMPLES

### Example 1

The following data in Table 1 was generated from a simulation conducted according to the methods of the present invention on the protein Caspase-3. Amino acids are listed on the left hand side, while the fragments are listed at the top. The binding affinities associated with the fragment-residue pairs are listed.

TABLE 1
Fragment Binding Affinity for Caspase-3

| | | | | | | | | | | tetra- | | |
|---|---|---|---|---|---|---|---|---|---|---|---|---|
| | acet- | | | carbox- | dimethyl | | imida- | iso- | pyrimi- | hydro- | | |
| | amide | acetone | benzene | ylic acid | sulfoxide | ethanol | zole | butane | dine | furan | urea | H₂O |
| ACE A 0 | 0 | 0 | 0 | 0 | 0 | 0 | 0 | 0 | 0 | 0 | 0 | 0 |
| ASN A 35 | 0 | −14.530 | 0 | 0 | −19.171 | 0 | −27.307 | 0 | 0 | −12.976 | −22.528 | 0 |
| SER A 36 | 0 | 0 | 0 | 0 | 0 | 0 | 0 | 0 | 0 | 0 | 0 | 0 |
| TYR A 37 | −21.098 | 0 | −6.808 | 0 | 0 | 0 | −21.307 | 0 | 0 | 0 | −22.528 | 0 |
| LYS A 38 | 0 | 0 | −6.808 | 0 | 0 | 0 | 0 | 0 | 0 | 0 | 0 | 0 |
| MET A 39 | 0 | 0 | 0 | 0 | 0 | 0 | 0 | 0 | 0 | 0 | 0 | 0 |
| ASP A 40 | 0 | 0 | 0 | 0 | 0 | 0 | 0 | 0 | 0 | 0 | 0 | 0 |
| TYR A 41 | 0 | 0 | 0 | 0 | 0 | 0 | 0 | 0 | −12.472 | 0 | 0 | 0 |
| PRO A 42 | 0 | 0 | 0 | 0 | 0 | 0 | 0 | 0 | 0 | 0 | 0 | 0 |
| GLU A 43 | 0 | 0 | 0 | −17.593 | 0 | −18.233 | 0 | 0 | 0 | 0 | −19.528 | −19 |
| MET A 44 | 0 | 0 | 0 | 0 | 0 | 0 | 0 | 0 | 0 | 0 | 0 | 0 |
| GLY A 45 | 0 | 0 | 0 | 0 | 0 | 0 | 0 | 0 | 0 | 0 | 0 | 0 |
| LEU A 46 | 0 | 0 | 0 | 0 | 0 | 0 | 0 | 0 | 0 | 0 | 0 | −18 |
| CYS A 47 | 0 | 0 | 0 | 0 | 0 | 0 | 0 | 0 | 0 | 0 | 0 | 0 |
| ILE A 48 | 0 | 0 | 0 | 0 | 0 | 0 | 0 | 0 | 0 | 0 | 0 | 0 |
| ILE A 49 | 0 | 0 | 0 | 0 | 0 | 0 | 0 | 0 | 0 | 0 | 0 | 0 |
| ILE A 50 | 0 | 0 | 0 | 0 | 0 | 0 | 0 | 0 | 0 | 0 | 0 | 0 |
| ASN A 51 | 0 | 0 | 0 | 0 | 0 | 0 | 0 | 0 | 0 | 0 | 0 | 0 |
| ASN A 52 | 0 | 0 | 0 | 0 | 0 | 0 | 0 | 0 | 0 | 0 | 0 | 0 |
| LYS A 53 | 0 | 0 | 0 | 0 | 0 | 0 | 0 | 0 | 0 | 0 | 0 | 0 |
| ASN A 54 | 0 | 0 | 0 | 0 | 0 | 0 | 0 | 0 | 0 | 0 | 0 | 0 |
| PHE A 55 | 0 | 0 | 0 | 0 | 0 | 0 | 0 | 0 | 0 | 0 | 0 | 0 |
| HIE A 56 | 0 | 0 | 0 | 0 | 0 | 0 | 0 | 0 | 0 | 0 | 0 | 0 |
| LYS A 57 | 0 | 0 | 0 | 0 | 0 | 0 | 0 | 0 | 0 | 0 | 0 | 0 |
| SER A 58 | 0 | 0 | 0 | 0 | 0 | 0 | 0 | 0 | 0 | 0 | 0 | 0 |
| THR A 59 | 0 | 0 | 0 | 0 | 0 | 0 | 0 | 0 | 0 | 0 | 0 | 0 |
| GLY A 60 | 0 | 0 | 0 | 0 | 0 | 0 | 0 | 0 | 0 | 0 | 0 | 0 |
| MET A 61 | −31.098 | −14.530 | 0 | −28.593 | −29.171 | −23.233 | −22.307 | 0 | 0 | −13.976 | −30.528 | 0 |
| THR A 62 | 0 | 0 | 0 | 0 | 0 | 0 | 0 | 0 | −11.472 | 0 | 0 | 0 |
| SER A 63 | 0 | 0 | 0 | −12.593 | 0 | 0 | 0 | 0 | −11.472 | 0 | 0 | 0 |
| ARG A 64 | −31.098 | −30.530 | −5.808 | −28.593 | −29.171 | −26.233 | −35.307 | −6.330 | −17.472 | −23.976 | −34.528 | −23 |
| SER A 65 | 0 | 0 | 0 | 0 | 0 | 0 | 0 | 0 | 0 | 0 | 0 | 0 |
| GLY A 66 | 0 | 0 | 0 | 0 | 0 | 0 | 0 | 0 | 0 | 0 | 0 | 0 |
| THR A 67 | 0 | 0 | 0 | 0 | 0 | 0 | 0 | 0 | 0 | 0 | 0 | −15 |
| ASP A 68 | 0 | 0 | 0 | 0 | 0 | 0 | 0 | 0 | 0 | 0 | 0 | 0 |
| VAL A 69 | 0 | 0 | 0 | 0 | 0 | 0 | 0 | 0 | 0 | 0 | 0 | 0 |
| ASP A 70 | 0 | 0 | 0 | 0 | 0 | 0 | 0 | 0 | 0 | 0 | 0 | −15 |
| ALA A 71 | 0 | 0 | 0 | 0 | 0 | 0 | 0 | 0 | 0 | 0 | 0 | 0 |
| ALA A 72 | 0 | 0 | 0 | 0 | 0 | 0 | 0 | 0 | 0 | 0 | 0 | 0 |
| ASN A 73 | 0 | 0 | 0 | 0 | 0 | 0 | 0 | 0 | 0 | 0 | 0 | 0 |
| LEU A 74 | 0 | 0 | 0 | 0 | 0 | 0 | 0 | 0 | 0 | 0 | 0 | 0 |
| ARG A 75 | 0 | 0 | 0 | 0 | 0 | 0 | 0 | 0 | 0 | 0 | 0 | 0 |
| GLU A 76 | 0 | 0 | 0 | 0 | 0 | 0 | 0 | 0 | 0 | 0 | 0 | 0 |
| THR A 77 | 0 | 0 | 0 | 0 | 0 | 0 | 0 | 0 | 0 | 0 | 0 | 0 |
| PHE A 78 | 0 | 0 | 0 | 0 | 0 | 0 | 0 | 0 | 0 | 0 | 0 | 0 |
| ARG A 79 | 0 | 0 | 0 | 0 | 0 | 0 | 0 | 0 | 0 | 0 | 0 | 0 |
| ASN A 80 | 0 | 0 | 0 | 0 | 0 | 0 | 0 | 0 | 0 | 0 | 0 | 0 |
| LEU A 81 | 0 | 0 | 0 | 0 | 0 | 0 | 0 | 0 | 0 | 0 | 0 | 0 |
| LYS A 82 | 0 | 0 | 0 | 0 | 0 | 0 | 0 | 0 | 0 | 0 | 0 | 0 |
| TYR A 83 | 0 | 0 | 0 | 0 | 0 | 0 | 0 | 0 | 0 | 0 | 0 | 0 |
| GLU A 84 | 0 | 0 | 0 | −12.593 | 0 | 0 | 0 | 0 | 0 | 0 | 0 | −18 |
| VAL A 85 | 0 | 0 | 0 | 0 | 0 | 0 | 0 | 0 | 0 | 0 | 0 | 0 |
| ARG A 86 | 0 | 0 | 0 | 0 | 0 | 0 | 0 | 0 | −10.472 | 0 | 0 | 0 |
| ASN A 87 | 0 | 0 | 0 | 0 | 0 | 0 | 0 | 0 | 0 | 0 | 0 | 0 |
| LYS A 88 | −27.098 | −16.530 | 0 | 0 | −21.171 | 0 | −30.307 | 0 | −12.472 | −12.976 | −26.528 | 0 |
| ASN A 89 | 0 | 0 | 0 | 0 | −16.171 | 0 | 0 | 0 | −12.472 | 0 | 0 | 0 |
| ASP A 90 | 0 | 0 | 0 | 0 | 0 | 0 | 0 | 0 | 0 | 0 | 0 | 0 |
| LEU A 91 | −27.098 | −15.530 | 0 | 0 | −17.171 | 0 | −30.307 | 0 | 0 | −11.976 | −26.528 | 0 |
| THR A 92 | 0 | 0 | 0 | 0 | 0 | 0 | 0 | 0 | 0 | 0 | 0 | 0 |
| ARG A 93 | 0 | 0 | 0 | 0 | 0 | 0 | 0 | 0 | −9.472 | 0 | 0 | 0 |
| GLU A 94 | 0 | 0 | 0 | −15.593 | −16.171 | −13.233 | 0 | 0 | 0 | 0 | 0 | 0 |
| GLU A 95 | −27.098 | −16.530 | 0 | 0 | −21.171 | 0 | −30.307 | 0 | 0 | −12.976 | −26.528 | 0 |
| ILE A 96 | 0 | 0 | 0 | 0 | 0 | 0 | 0 | 0 | 0 | 0 | 0 | 0 |
| VAL A 97 | −18.098 | −17.530 | 0 | 0 | −24.171 | 0 | 0 | 0 | 0 | 0 | −17.528 | 0 |
| GLU A 98 | −22.098 | −13.530 | 0 | −21.593 | −17.171 | −23.233 | −30.307 | 0 | 0 | 0 | −25.528 | −27 |
| LEU A 99 | 0 | 0 | 0 | 0 | −16.171 | 0 | −23.307 | 0 | −10.472 | 0 | 0 | 0 |
| MET A 100 | 0 | 0 | 0 | 0 | 0 | 0 | 0 | 0 | 0 | 0 | 0 | 0 |
| ARG A 101 | −18.098 | −17.530 | 0 | −15.593 | −24.171 | −23.233 | 0 | 0 | 0 | −9.976 | −25.528 | −27 |
| ASP A 102 | −21.098 | 0 | 0 | −21.593 | 0 | −23.233 | −30.307 | 0 | 0 | 0 | −25.528 | −27 |
| VAL A 103 | 0 | 0 | 0 | 0 | 0 | 0 | 0 | 0 | 0 | 0 | 0 | 0 |
| SER A 104 | 0 | −16.530 | 0 | 0 | −18.171 | 0 | −24.307 | 0 | −10.472 | −14.976 | 0 | 0 |
| LYS A 105 | −22.098 | −16.530 | 0 | −21.593 | −18.171 | −23.233 | −30.307 | 0 | −10.472 | −14.976 | −25.528 | −27 |
| GLU A 106 | −21.098 | −14.530 | 0 | 0 | −18.171 | −12.233 | −24.307 | 0 | −10.472 | −14.976 | 0 | 0 |
| ASP A 107 | 0 | −16.530 | −5.808 | −16.593 | −18.171 | −18.233 | −24.307 | 0 | 0 | −14.976 | −19.528 | 0 |
| HIE A 108 | 0 | 0 | 0 | 0 | 0 | 0 | 0 | 0 | 0 | 0 | 0 | 0 |
| SER A 109 | 0 | 0 | 0 | 0 | 0 | 0 | 0 | 0 | 0 | 0 | 0 | 0 |
| LYS A 110 | 0 | 0 | 0 | −17.593 | 0 | −18.233 | 0 | 0 | 0 | 0 | −19.528 | −17 |
| ARG A 111 | 0 | 0 | 0 | −17.593 | 0 | −18.233 | 0 | 0 | 0 | 0 | −19.528 | −19 |
| SER A 112 | 0 | 0 | 0 | 0 | 0 | 0 | 0 | 0 | 0 | 0 | 0 | 0 |
| SER A 113 | 0 | 0 | 0 | 0 | 0 | 0 | 0 | 0 | 0 | 0 | 0 | 0 |
| PHE A 114 | 0 | 0 | 0 | 0 | 0 | 0 | 0 | 0 | 0 | 0 | 0 | 0 |
| VAL A 115 | 0 | 0 | 0 | 0 | 0 | 0 | 0 | 0 | 0 | 0 | 0 | 0 |
| CYS A 116 | 0 | 0 | 0 | 0 | 0 | 0 | 0 | 0 | 0 | 0 | 0 | 0 |
| VAL A 117 | 0 | 0 | 0 | 0 | 0 | 0 | 0 | 0 | 0 | 0 | 0 | 0 |
| LEU A 118 | 0 | 0 | 0 | 0 | 0 | 0 | 0 | 0 | 0 | 0 | 0 | 0 |
| LEU A 119 | 0 | 0 | 0 | 0 | 0 | 0 | 0 | 0 | 0 | 0 | 0 | −15 |
| SER A 120 | −26.098 | −14.530 | −5.808 | −28.593 | −27.171 | −26.233 | −35.307 | −6.330 | −17.472 | −23.976 | −34.528 | −23 |
| HIP A 121 | −31.098 | −20.530 | −5.808 | −28.593 | −29.171 | −23.233 | −35.307 | −6.330 | −17.472 | −23.976 | −30.528 | −26 |
| GLY A 122 | −31.098 | −20.530 | 0 | −28.593 | −29.171 | −23.233 | −22.307 | 0 | −14.472 | −14.976 | −30.528 | −20 |
| GLU A 123 | 0 | −19.530 | 0 | 0 | −23.171 | 0 | 0 | 0 | 0 | 0 | 0 | 0 |
| GLU A 124 | 0 | 0 | 0 | 0 | 0 | 0 | 0 | 0 | −13.472 | 0 | 0 | 0 |
| GLY A 125 | 0 | 0 | 0 | 0 | 0 | 0 | 0 | 0 | 0 | 0 | 0 | 0 |
| ILE A 126 | 0 | 0 | 0 | 0 | 0 | 0 | 0 | 0 | 0 | 0 | 0 | 0 |
| ILE A 127 | 0 | 0 | 0 | 0 | 0 | 0 | 0 | 0 | 0 | 0 | 0 | 0 |
| PHE A 128 | 0 | −19.530 | 0 | 0 | −29.171 | 0 | 0 | 0 | 0 | 0 | 0 | 0 |
| GLY A 129 | 0 | 0 | 0 | 0 | 0 | 0 | 0 | 0 | 0 | 0 | 0 | 0 |
| THR A 130 | 0 | 0 | 0 | 0 | 0 | 0 | 0 | 0 | 0 | 0 | 0 | 0 |
| ASN A 131 | 0 | 0 | 0 | 0 | 0 | 0 | 0 | 0 | 0 | 0 | 0 | 0 |
| GLY A 132 | 0 | 0 | 0 | 0 | 0 | 0 | 0 | 0 | 0 | 0 | 0 | 0 |
| PRO A 133 | 0 | 0 | 0 | 0 | 0 | 0 | 0 | 0 | −9.472 | 0 | 0 | 0 |
| VAL A 134 | 0 | 0 | 0 | 0 | 0 | 0 | 0 | 0 | −9.472 | 0 | 0 | 0 |
| ASP A 135 | 0 | 0 | 0 | 0 | 0 | 0 | 0 | 0 | −9.472 | 0 | 0 | 0 |
| LEU A 136 | 0 | 0 | 0 | 0 | 0 | 0 | 0 | 0 | 0 | 0 | 0 | 0 |
| LYS A 137 | 0 | 0 | −7.808 | −13.593 | 0 | −11.233 | −25.307 | 0 | 0 | 0 | −23.528 | −21 |
| LYS A 138 | −18.098 | −17.530 | −5.808 | 0 | −24.171 | 0 | 0 | 0 | 0 | −9.976 | −17.528 | 0 |
| ILE A 139 | 0 | 0 | 0 | 0 | 0 | 0 | 0 | 0 | 0 | 0 | 0 | 0 |
| THR A 140 | 0 | 0 | 0 | 0 | 0 | 0 | 0 | 0 | 0 | 0 | 0 | 0 |
| ASN A 141 | 0 | 0 | −5.808 | 0 | −16.171 | 0 | −25.307 | 0 | 0 | 0 | −23.528 | 0 |
| PHE A 142 | 0 | 0 | −5.808 | 0 | 0 | 0 | 0 | 0 | 0 | 0 | 0 | 0 |
| PHE A 143 | 0 | 0 | 0 | 0 | 0 | 0 | 0 | 0 | 0 | 0 | 0 | 0 |
| ARG A 144 | −24.098 | −13.530 | −5.808 | 0 | −16.171 | −15.233 | −21.307 | 0 | 0 | −9.976 | −18.528 | −18 |
| GLY A 145 | 0 | 0 | 0 | 0 | 0 | 0 | 0 | 0 | 0 | 0 | 0 | 0 |
| ASP A 146 | 0 | 0 | 0 | −14.593 | 0 | −13.233 | 0 | 0 | 0 | 0 | 0 | 0 |
| ARG A 147 | 0 | −13.530 | −5.808 | −14.593 | −16.171 | −13.233 | 0 | 0 | 0 | 0 | 0 | 0 |
| CYS A 148 | 0 | 0 | 0 | 0 | −18.171 | −12.233 | −24.307 | 0 | 0 | −14.976 | 0 | 0 |
| ARG A 149 | −21.098 | −16.530 | 0 | 0 | −18.171 | −12.233 | −24.307 | 0 | −10.472 | −14.976 | 0 | 0 |
| SER A 150 | −21.098 | −16.530 | 0 | 0 | −18.171 | −12.233 | −24.307 | 0 | −10.472 | −14.976 | 0 | 0 |
| LEU A 151 | 0 | 0 | 0 | 0 | 0 | 0 | 0 | 0 | 0 | 0 | 0 | 0 |
| THR A 152 | 0 | 0 | 0 | 0 | 0 | 0 | 0 | 0 | 0 | 0 | 0 | −19 |
| GLY A 153 | −21.098 | 0 | −6.808 | 0 | 0 | −12.233 | −22.307 | 0 | −13.472 | −11.976 | −22.528 | −19 |
| LYS A 154 | 0 | 0 | 0 | 0 | 0 | 0 | 0 | 0 | 0 | 0 | 0 | 0 |
| PRO A 155 | 0 | 0 | 0 | 0 | 0 | 0 | 0 | 0 | 0 | 0 | 0 | 0 |
| LYS A 156 | 0 | 0 | 0 | 0 | 0 | 0 | 0 | 0 | 0 | 0 | 0 | −19 |
| LEU A 157 | 0 | 0 | 0 | 0 | 0 | 0 | 0 | 0 | 0 | 0 | 0 | 0 |
| PHE A 158 | 0 | 0 | 0 | 0 | 0 | 0 | 0 | 0 | 0 | 0 | 0 | 0 |
| ILE A 159 | 0 | 0 | 0 | 0 | 0 | 0 | 0 | 0 | 0 | 0 | 0 | 0 |
| ILE A 160 | 0 | 0 | 0 | 0 | 0 | 0 | 0 | 0 | 0 | 0 | 0 | 0 |
| GLN A 161 | −26.098 | −13.530 | −5.808 | 0 | −16.171 | 0 | −29.307 | −6.330 | 0 | −23.976 | −26.528 | −15 |
| ALA A 162 | −31.098 | −30.530 | −5.808 | −28.593 | −29.171 | −26.233 | −29.307 | −6.330 | 0 | −23.976 | −34.528 | 0 |
| CYM A 163 | −31.098 | −30.530 | −5.808 | −28.593 | −29.171 | −23.233 | −35.307 | −6.330 | −17.472 | −23.976 | −34.528 | −26 |
| ARG A 164 | 0 | 0 | 0 | 0 | 0 | 0 | 0 | 0 | −14.472 | 0 | 0 | 0 |
| GLY A 165 | 0 | 0 | 0 | 0 | 0 | 0 | 0 | 0 | 0 | 0 | 0 | 0 |
| THR A 166 | 0 | 0 | 0 | 0 | 0 | 0 | 0 | 0 | 0 | 0 | 0 | 0 |
| GLU A 167 | −26.098 | −16.530 | 0 | 0 | −25.171 | 0 | −30.307 | 0 | −10.472 | −10.976 | −25.528 | 0 |
| LEU A 168 | 0 | 0 | 0 | 0 | −25.171 | 0 | 0 | 0 | 0 | −10.976 | 0 | 0 |
| ASP A 169 | 0 | 0 | 0 | 0 | 0 | 0 | 0 | 0 | 0 | 0 | 0 | 0 |
| CYS A 170 | 0 | 0 | 0 | 0 | 0 | 0 | 0 | 0 | 0 | 0 | 0 | 0 |
| GLY A 171 | 0 | 0 | 0 | 0 | 0 | 0 | 0 | 0 | 0 | 0 | 0 | 0 |
| ILE A 172 | 0 | 0 | 0 | 0 | 0 | 0 | 0 | 0 | 0 | 0 | 0 | 0 |
| GLU A 173 | 0 | 0 | 0 | 0 | 0 | 0 | 0 | 0 | 0 | 0 | 0 | 0 |
| NME A 999 | 0 | 0 | 0 | 0 | 0 | 0 | 0 | 0 | 0 | 0 | 0 | 0 |
| ACE E 0 | 0 | 0 | 0 | 0 | 0 | 0 | 0 | 0 | 0 | 0 | 0 | 0 |
| HID E 185 | 0 | −15.530 | 0 | −19.593 | −18.171 | 0 | −19.307 | 0 | 0 | 0 | −18.528 | −16 |
| LYS E 186 | 0 | 0 | 0 | −15.593 | −18.171 | −12.233 | 0 | 0 | 0 | 0 | 0 | −16 |
| ILE E 187 | −21.098 | −15.530 | 0 | 0 | −20.171 | 0 | −22.307 | 0 | −13.472 | −11.976 | −23.528 | 0 |
| PRO E 188 | 0 | 0 | 0 | 0 | 0 | 0 | 0 | 0 | 0 | 0 | 0 | 0 |
| VAL E 189 | 0 | 0 | −5.808 | 0 | 0 | 0 | 0 | 0 | 0 | 0 | 0 | 0 |
| ASP E 190 | 0 | 0 | −7.808 | 0 | 0 | −11.233 | 0 | 0 | 0 | 0 | 0 | 0 |
| ALA E 191 | 0 | 0 | 0 | 0 | 0 | 0 | 0 | 0 | 0 | 0 | 0 | −19 |
| ASP E 192 | −21.098 | 0 | 0 | 0 | 0 | 0 | −21.307 | 0 | −13.472 | 0 | −22.528 | −19 |
| PHE E 193 | 0 | 0 | 0 | 0 | 0 | 0 | 0 | 0 | 0 | 0 | 0 | 0 |
| LEU E 194 | 0 | 0 | 0 | 0 | 0 | 0 | 0 | 0 | 0 | 0 | 0 | 0 |
| TYR E 195 | 0 | 0 | −7.808 | 0 | 0 | −11.233 | 0 | 0 | 0 | 0 | 0 | 0 |
| ALA E 196 | 0 | 0 | 0 | 0 | 0 | 0 | 0 | 0 | 0 | 0 | 0 | 0 |
| TYR E 197 | 0 | 0 | 0 | 0 | 0 | 0 | 0 | 0 | 0 | 0 | 0 | 0 |
| SER E 198 | 0 | 0 | 0 | 0 | 0 | 0 | 0 | 0 | 0 | 0 | 0 | 0 |
| THR E 199 | 0 | 0 | 0 | 0 | 0 | 0 | 0 | 0 | 0 | 0 | 0 | 0 |
| ALA E 200 | 0 | 0 | 0 | 0 | 0 | 0 | 0 | 0 | 0 | 0 | 0 | 0 |
| PRO E 201 | 0 | 0 | 0 | 0 | 0 | 0 | 0 | 0 | −14.472 | 0 | 0 | 0 |
| GLY E 202 | 0 | 0 | 0 | 0 | 0 | 0 | 0 | 0 | 0 | 0 | 0 | 0 |
| TYR E 203 | 0 | 0 | 0 | 0 | 0 | 0 | 0 | 0 | 0 | −9.976 | 0 | 0 |
| TYR E 204 | 0 | 0 | 0 | −12.593 | −29.171 | 0 | 0 | 0 | −11.472 | −12.976 | 0 | 0 |
| SER E 205 | −31.098 | −30.530 | 0 | −28.593 | −29.171 | −26.233 | −35.307 | −6.330 | −17.472 | −23.976 | −34.528 | −23 |
| TRP E 206 | −26.098 | 0 | 0 | 0 | 0 | 0 | 0 | −6.330 | −11.472 | 0 | −26.528 | 0 |
| ARG E 207 | −31.098 | −30.530 | −5.808 | −28.593 | −29.171 | −26.233 | −35.307 | −6.330 | −17.472 | −23.976 | −34.528 | −15 |
| ASN E 208 | 0 | 0 | 0 | 0 | 0 | 0 | 0 | 0 | 0 | −9.976 | 0 | 0 |
| SER E 209 | 0 | 0 | 0 | 0 | 0 | 0 | 0 | 0 | 0 | 0 | 0 | 0 |
| LYS E 210 | 0 | 0 | 0 | −13.593 | 0 | 0 | 0 | 0 | 0 | 0 | 0 | 0 |
| ASP E 211 | 0 | 0 | 0 | −13.593 | 0 | 0 | 0 | 0 | 0 | 0 | 0 | 0 |
| GLY E 212 | 0 | 0 | 0 | 0 | 0 | 0 | 0 | 0 | 0 | 0 | 0 | 0 |
| SER E 213 | 0 | 0 | 0 | 0 | 0 | 0 | 0 | 0 | 0 | 0 | 0 | 0 |
| TRP E 214 | 0 | 0 | 0 | 0 | 0 | 0 | 0 | 0 | 0 | −9.976 | 0 | 0 |
| PHE E 215 | 0 | 0 | 0 | 0 | 0 | 0 | 0 | 0 | 0 | 0 | 0 | 0 |
| ILE E 216 | 0 | 0 | 0 | 0 | 0 | 0 | 0 | 0 | 0 | 0 | 0 | 0 |
| GLN E 217 | 0 | 0 | 0 | 0 | 0 | 0 | 0 | 0 | 0 | 0 | 0 | 0 |
| SER E 218 | 0 | 0 | 0 | 0 | 0 | 0 | 0 | 0 | 0 | 0 | 0 | 0 |
| LEU E 219 | 0 | 0 | 0 | 0 | 0 | 0 | 0 | 0 | 0 | 0 | 0 | 0 |
| CYS E 220 | 0 | 0 | 0 | 0 | 0 | 0 | 0 | 0 | 0 | 0 | 0 | 0 |
| ALA E 221 | 0 | 0 | 0 | 0 | 0 | 0 | 0 | 0 | 0 | 0 | 0 | 0 |
| MET E 222 | 0 | 0 | 0 | 0 | 0 | 0 | 0 | 0 | 0 | 0 | 0 | 0 |
| LEU E 223 | 0 | 0 | 0 | 0 | 0 | 0 | 0 | 0 | 0 | 0 | 0 | 0 |
| LYS E 224 | 0 | 0 | 0 | 0 | 0 | 0 | 0 | 0 | 0 | 0 | 0 | 0 |
| GLN E 225 | 0 | 0 | 0 | 0 | 0 | 0 | −29.307 | 0 | −11.472 | 0 | 0 | −18 |
| TYR E 226 | 0 | 0 | 0 | 0 | 0 | 0 | −29.307 | 0 | −11.472 | 0 | 0 | −18 |
| ALA E 227 | 0 | 0 | 0 | 0 | 0 | 0 | 0 | 0 | 0 | 0 | 0 | 0 |
| ASP E 228 | 0 | 0 | 0 | 0 | 0 | 0 | −29.307 | 0 | −11.472 | 0 | 0 | −18 |
| LYS E 229 | 0 | 0 | 0 | 0 | 0 | 0 | −29.307 | 0 | −12.472 | 0 | 0 | −18 |
| LEU E 230 | −18.098 | −13.530 | 0 | −16.593 | −17.171 | −15.233 | −21.307 | 0 | −12.472 | 0 | −20.528 | −16 |
| GLU E 231 | 0 | 0 | 0 | 0 | 0 | 0 | 0 | 0 | 0 | 0 | 0 | 0 |
| PHE E 232 | 0 | 0 | 0 | 0 | 0 | 0 | 0 | 0 | 0 | 0 | 0 | 0 |
| MET E 233 | 0 | 0 | 0 | 0 | 0 | 0 | 0 | 0 | 0 | 0 | 0 | 0 |
| HIE E 234 | 0 | 0 | 0 | −16.593 | 0 | 0 | 0 | 0 | 0 | 0 | 0 | −26 |
| ILE E 235 | 0 | 0 | 0 | 0 | 0 | 0 | 0 | 0 | 0 | 0 | 0 | 0 |
| LEU E 236 | 0 | 0 | 0 | 0 | 0 | 0 | 0 | 0 | 0 | 0 | 0 | 0 |
| THR E 237 | 0 | 0 | 0 | 0 | 0 | 0 | 0 | 0 | 0 | 0 | 0 | 0 |
| ARG E 238 | −18.098 | −13.530 | 0 | −16.593 | −17.171 | −15.233 | −22.307 | 0 | −12.472 | −9.976 | −20.528 | −26 |
| VAL E 239 | 0 | 0 | 0 | 0 | 0 | 0 | 0 | 0 | 0 | 0 | 0 | 0 |
| ASN E 240 | 0 | 0 | 0 | 0 | 0 | 0 | 0 | 0 | 0 | 0 | 0 | 0 |
| ARG E 241 | −22.098 | −16.530 | −5.808 | 0 | −20.171 | 0 | −24.307 | −4.330 | −12.472 | −15.976 | −19.528 | −23 |
| LYS E 242 | −18.098 | 0 | 0 | 0 | 0 | 0 | −24.307 | 0 | −9.472 | 0 | −19.528 | 0 |
| VAL E 243 | 0 | 0 | 0 | 0 | 0 | 0 | 0 | 0 | 0 | 0 | 0 | 0 |
| ALA E 244 | 0 | 0 | 0 | 0 | 0 | 0 | 0 | 0 | 0 | 0 | 0 | 0 |
| THR E 245 | 0 | −15.530 | 0 | −16.593 | −20.171 | 0 | 0 | −4.330 | −11.472 | −15.976 | 0 | 0 |
| GLU E 246 | −22.098 | −16.530 | 0 | 0 | −19.171 | 0 | −24.307 | 0 | −12.472 | 0 | −19.528 | 0 |
| PHE E 247 | 0 | 0 | 0 | 0 | 0 | 0 | 0 | 0 | 0 | 0 | 0 | 0 |
| GLU E 248 | 0 | 0 | 0 | −16.593 | 0 | 0 | 0 | 0 | 0 | −9.976 | 0 | 0 |
| SER E 249 | 0 | 0 | 0 | 0 | 0 | 0 | 0 | 0 | 0 | −9.976 | 0 | 0 |
| PHE E 250 | 0 | 0 | 0 | 0 | 0 | 0 | 0 | 0 | 0 | 0 | 0 | 0 |
| SER E 251 | 0 | 0 | 0 | 0 | 0 | 0 | 0 | 0 | 0 | 0 | 0 | 0 |
| PHE E 252 | 0 | 0 | 0 | 0 | 0 | 0 | 0 | 0 | 0 | 0 | 0 | 0 |
| ASP E 253 | 0 | 0 | 0 | 0 | 0 | 0 | 0 | 0 | 0 | 0 | 0 | 0 |
| ALA E 254 | 0 | 0 | 0 | 0 | 0 | 0 | 0 | 0 | 0 | 0 | 0 | 0 |
| THR E 255 | 0 | 0 | 0 | 0 | 0 | 0 | 0 | 0 | 0 | 0 | 0 | 0 |
| PHE E 256 | 0 | 0 | 0 | 0 | 0 | 0 | 0 | 0 | 0 | 0 | 0 | 0 |
| HIE E 257 | 0 | 0 | 0 | 0 | 0 | 0 | 0 | 0 | 0 | 0 | 0 | 0 |
| ALA E 258 | 0 | 0 | 0 | 0 | 0 | 0 | 0 | 0 | 0 | 0 | 0 | 0 |
| LYS E 259 | 0 | 0 | 0 | 0 | 0 | 0 | 0 | 0 | 0 | 0 | 0 | 0 |
| LYS E 260 | 0 | 0 | 0 | −16.593 | 0 | 0 | 0 | 0 | 0 | 0 | 0 | 0 |
| GLN E 261 | 0 | 0 | 0 | 0 | 0 | 0 | 0 | 0 | 0 | 0 | 0 | 0 |
| ILE E 262 | 0 | 0 | 0 | 0 | 0 | 0 | 0 | 0 | 0 | 0 | 0 | 0 |
| PRO E 263 | 0 | 0 | 0 | 0 | 0 | 0 | 0 | 0 | 0 | 0 | 0 | 0 |
| CYS E 264 | 0 | 0 | 0 | 0 | 0 | 0 | 0 | 0 | 0 | 0 | 0 | 0 |
| ILE E 265 | 0 | 0 | 0 | 0 | 0 | 0 | 0 | 0 | 0 | 0 | 0 | 0 |
| VAL E 266 | 0 | 0 | 0 | 0 | 0 | 0 | 0 | 0 | 0 | 0 | 0 | 0 |
| SER E 267 | 0 | 0 | 0 | 0 | 0 | 0 | 0 | 0 | 0 | 0 | 0 | 0 |
| MET E 268 | 0 | 0 | −7.808 | 0 | 0 | −11.233 | 0 | 0 | 0 | 0 | 0 | 0 |
| LEU E 269 | 0 | 0 | 0 | 0 | 0 | 0 | 0 | 0 | 0 | 0 | 0 | 0 |
| THR E 270 | −21.098 | 0 | −6.808 | 0 | 0 | 0 | −21.307 | 0 | −13.472 | 0 | −22.528 | 0 |
| LYS E 271 | −21.098 | −15.530 | −6.808 | 0 | −20.171 | −12.233 | −22.307 | 0 | −13.472 | −11.976 | −22.528 | 0 |
| GLU E 272 | −25.098 | −18.530 | 0 | −19.593 | −23.171 | −22.233 | −27.307 | 0 | −13.472 | −12.976 | −22.528 | −21 |
| LEU E 273 | 0 | 0 | 0 | 0 | 0 | 0 | 0 | 0 | 0 | 0 | 0 | 0 |
| TYR E 274 | 0 | 0 | 0 | 0 | 0 | 0 | 0 | 0 | 0 | 0 | 0 | 0 |
| PHE E 275 | 0 | 0 | 0 | 0 | 0 | 0 | 0 | 0 | 0 | 0 | 0 | 0 |
| TYR E 276 | 0 | 0 | 0 | 0 | 0 | 0 | 0 | −4.330 | 0 | 0 | 0 | 0 |
| HIE E 277 | 0 | 0 | 0 | 0 | 0 | 0 | 0 | −4.330 | 0 | 0 | 0 | 0 |
| NME E 999 | 0 | 0 | 0 | 0 | 0 | 0 | 0 | 0 | 0 | 0 | 0 | 0 |
| ACE B 0 | −18.098 | −13.530 | 0 | −16.593 | −17.171 | −15.233 | −21.307 | 0 | −12.472 | 0 | −20.528 | −16 |
| ASN B 35 | 0 | 0 | 0 | 0 | 0 | 0 | 0 | 0 | 0 | 0 | 0 | −23 |
| SER B 36 | 0 | 0 | 0 | 0 | 0 | 0 | 0 | 0 | 0 | 0 | 0 | 0 |
| TYR B 37 | −23.098 | 0 | 0 | 0 | 0 | 0 | −27.307 | 0 | 0 | 0 | 0 | 0 |
| LYS B 38 | 0 | 0 | 0 | 0 | 0 | 0 | 0 | 0 | 0 | 0 | 0 | 0 |
| MET B 39 | 0 | 0 | 0 | 0 | 0 | 0 | 0 | 0 | 0 | 0 | 0 | 0 |
| ASP B 40 | 0 | 0 | 0 | 0 | 0 | 0 | 0 | 0 | 0 | 0 | 0 | 0 |
| TYR B 41 | 0 | 0 | 0 | 0 | 0 | 0 | 0 | 0 | 0 | 0 | 0 | 0 |
| PRO B 42 | 0 | 0 | 0 | 0 | 0 | 0 | 0 | 0 | 0 | 0 | 0 | 0 |
| GLU B 43 | 0 | 0 | 0 | 0 | 0 | 0 | −22.307 | 0 | 0 | 0 | 0 | −17 |
| MET B 44 | 0 | 0 | 0 | 0 | 0 | 0 | 0 | 0 | 0 | 0 | 0 | 0 |
| GLY B 45 | 0 | 0 | 0 | 0 | 0 | 0 | 0 | 0 | 0 | 0 | 0 | −17 |
| LEU B 46 | 0 | 0 | 0 | 0 | 0 | 0 | 0 | 0 | 0 | 0 | 0 | 0 |
| CYS B 47 | 0 | 0 | 0 | 0 | 0 | 0 | 0 | 0 | 0 | 0 | 0 | 0 |
| ILE B 48 | 0 | 0 | 0 | 0 | 0 | 0 | 0 | 0 | 0 | 0 | 0 | 0 |
| ILE B 49 | 0 | 0 | 0 | 0 | 0 | 0 | 0 | 0 | 0 | 0 | 0 | 0 |
| ILE B 50 | 0 | 0 | 0 | 0 | 0 | 0 | 0 | 0 | 0 | 0 | 0 | 0 |
| ASN B 51 | 0 | 0 | 0 | 0 | 0 | 0 | 0 | 0 | 0 | 0 | 0 | 0 |
| ASN B 52 | 0 | 0 | 0 | 0 | 0 | 0 | 0 | 0 | 0 | 0 | 0 | 0 |
| LYS B 53 | −22.098 | 0 | 0 | −15.593 | −20.171 | −14.233 | 0 | 0 | 0 | −11.976 | −17.528 | 0 |
| ASN B 54 | 0 | 0 | 0 | 0 | 0 | −11.233 | 0 | 0 | 0 | 0 | 0 | 0 |
| PHE B 55 | 0 | 0 | 0 | 0 | 0 | 0 | 0 | 0 | 0 | 0 | 0 | 0 |
| HIE B 56 | 0 | 0 | 0 | 0 | 0 | 0 | 0 | 0 | 0 | 0 | 0 | 0 |
| LYS B 57 | 0 | 0 | 0 | 0 | 0 | 0 | 0 | 0 | 0 | 0 | 0 | 0 |
| SER B 58 | 0 | 0 | 0 | 0 | 0 | 0 | 0 | 0 | 0 | 0 | 0 | 0 |
| THR B 59 | 0 | 0 | 0 | 0 | 0 | 0 | 0 | 0 | 0 | 0 | 0 | 0 |
| GLY B 60 | 0 | 0 | 0 | 0 | 0 | 0 | 0 | 0 | 0 | 0 | 0 | 0 |
| MET B 61 | −27.098 | 0 | 0 | 0 | −29.171 | −22.233 | −31.307 | −4.330 | −16.472 | −19.976 | 0 | 0 |
| THR B 62 | 0 | 0 | 0 | 0 | 0 | 0 | 0 | 0 | −12.472 | 0 | 0 | 0 |
| SER B 63 | 0 | 0 | 0 | 0 | 0 | −11.233 | 0 | 0 | 0 | 0 | 0 | 0 |
| ARG B 64 | −27.098 | −16.530 | 0 | −21.593 | −29.171 | −18.233 | −35.307 | −4.330 | −29.472 | −19.976 | −34.528 | −21 |
| SER B 65 | −22.098 | 0 | 0 | −15.593 | −20.171 | −14.233 | 0 | 0 | 0 | −11.976 | −17.528 | 0 |
| GLY B 66 | −22.098 | 0 | 0 | −15.593 | −20.171 | −14.233 | 0 | 0 | 0 | −11.976 | −17.528 | 0 |
| THR B 67 | 0 | 0 | 0 | 0 | 0 | 0 | 0 | 0 | 0 | 0 | 0 | 0 |
| ASP B 68 | −22.098 | 0 | 0 | −15.593 | −20.171 | −14.233 | 0 | 0 | 0 | −11.976 | −17.528 | 0 |
| VAL B 69 | 0 | 0 | 0 | 0 | 0 | 0 | 0 | 0 | 0 | 0 | 0 | 0 |
| ASP B 70 | 0 | 0 | 0 | 0 | 0 | 0 | 0 | 0 | 0 | 0 | 0 | 0 |
| ALA B 71 | 0 | 0 | 0 | 0 | 0 | 0 | 0 | 0 | 0 | 0 | 0 | 0 |
| ALA B 72 | 0 | 0 | 0 | 0 | 0 | 0 | 0 | 0 | 0 | 0 | 0 | 0 |
| ASN B 73 | 0 | 0 | 0 | 0 | 0 | 0 | 0 | 0 | 0 | 0 | 0 | 0 |
| LEU B 74 | 0 | 0 | 0 | 0 | 0 | 0 | 0 | 0 | 0 | 0 | 0 | 0 |
| ARG B 75 | 0 | 0 | 0 | 0 | 0 | 0 | 0 | 0 | 0 | 0 | 0 | 0 |
| GLU B 76 | 0 | 0 | 0 | 0 | 0 | 0 | 0 | 0 | 0 | 0 | 0 | 0 |
| THR B 77 | 0 | 0 | 0 | 0 | 0 | 0 | 0 | 0 | 0 | 0 | 0 | 0 |
| PHE B 78 | 0 | 0 | 0 | 0 | 0 | 0 | 0 | 0 | 0 | 0 | 0 | 0 |
| ARG B 79 | 0 | 0 | 0 | 0 | 0 | 0 | 0 | 0 | 0 | 0 | 0 | 0 |
| ASN B 80 | 0 | 0 | 0 | 0 | 0 | 0 | 0 | 0 | 0 | 0 | 0 | 0 |
| LEU B 81 | 0 | 0 | 0 | 0 | 0 | 0 | 0 | 0 | 0 | 0 | 0 | 0 |
| LYS B 82 | 0 | 0 | 0 | 0 | 0 | 0 | 0 | 0 | 0 | 0 | 0 | 0 |
| TYR B 83 | 0 | 0 | 0 | 0 | 0 | 0 | 0 | 0 | 0 | 0 | 0 | 0 |
| GLU B 84 | 0 | 0 | 0 | 0 | 0 | 0 | 0 | 0 | 0 | 0 | 0 | −17 |
| VAL B 85 | 0 | 0 | 0 | 0 | 0 | 0 | 0 | 0 | 0 | 0 | 0 | 0 |
| ARG B 86 | 0 | −15.530 | 0 | 0 | −21.171 | 0 | 0 | 0 | 0 | −9.976 | 0 | 0 |
| ASN B 87 | 0 | 0 | 0 | 0 | 0 | 0 | 0 | 0 | 0 | 0 | 0 | 0 |
| LYS B 88 | 0 | −15.530 | 0 | 0 | −21.171 | 0 | −19.307 | 0 | 0 | −9.976 | 0 | 0 |
| ASN B 89 | 0 | 0 | 0 | 0 | 0 | 0 | 0 | 0 | 0 | 0 | 0 | 0 |
| ASP B 90 | 0 | 0 | 0 | 0 | 0 | 0 | 0 | 0 | 0 | 0 | 0 | 0 |
| LEU B 91 | 0 | 0 | 0 | 0 | 0 | 0 | −19.307 | 0 | 0 | 0 | 0 | 0 |
| THR B 92 | 0 | 0 | 0 | 0 | 0 | 0 | 0 | 0 | 0 | 0 | 0 | 0 |
| ARG B 93 | −22.098 | 0 | 0 | 0 | −20.171 | 0 | −29.307 | 0 | 0 | 0 | −18.528 | −17 |
| GLU B 94 | −22.098 | −19.530 | −5.808 | −13.593 | −25.171 | 0 | −29.307 | 0 | −9.472 | −13.976 | −23.528 | −16 |
| GLU B 95 | 0 | 0 | 0 | 0 | 0 | 0 | −19.307 | 0 | 0 | 0 | 0 | 0 |
| ILE B 96 | 0 | 0 | 0 | 0 | 0 | 0 | 0 | 0 | 0 | 0 | 0 | 0 |
| VAL B 97 | −23.098 | −19.530 | 0 | −13.593 | −25.171 | 0 | −30.307 | 0 | −16.472 | −13.976 | −19.528 | −16 |
| GLU B 98 | −23.098 | −19.530 | 0 | −15.593 | −25.171 | 0 | −30.307 | 0 | −16.472 | 0 | −19.528 | 0 |
| LEU B 99 | 0 | −13.530 | 0 | 0 | −20.171 | 0 | 0 | 0 | 0 | −9.976 | 0 | 0 |
| MET B 100 | 0 | 0 | 0 | 0 | 0 | 0 | 0 | 0 | 0 | 0 | 0 | 0 |
| ARG B 101 | −22.098 | 0 | 0 | −15.593 | 0 | 0 | −30.307 | 0 | −16.472 | 0 | −19.528 | 0 |
| ASP B 102 | −22.098 | 0 | 0 | −15.593 | 0 | 0 | 0 | 0 | 0 | 0 | −18.528 | 0 |
| VAL B 103 | 0 | 0 | 0 | 0 | 0 | 0 | 0 | 0 | 0 | 0 | 0 | 0 |
| SER B 104 | 0 | 0 | 0 | 0 | 0 | 0 | 0 | 0 | 0 | 0 | 0 | 0 |
| LYS B 105 | −22.098 | 0 | 0 | −15.593 | 0 | 0 | 0 | 0 | 0 | 0 | −18.528 | 0 |
| GLU B 106 | 0 | 0 | 0 | 0 | 0 | 0 | 0 | 0 | 0 | 0 | 0 | 0 |
| ASP B 107 | 0 | 0 | 0 | 0 | 0 | 0 | −21.307 | 0 | 0 | 0 | 0 | 0 |
| HIE B 108 | 0 | 0 | 0 | 0 | 0 | 0 | 0 | 0 | 0 | 0 | 0 | 0 |
| SER B 109 | 0 | 0 | 0 | 0 | 0 | 0 | 0 | 0 | 0 | 0 | 0 | 0 |
| LYS B 110 | 0 | 0 | 0 | 0 | 0 | 0 | −22.307 | 0 | 0 | 0 | 0 | 0 |
| ARG B 111 | 0 | 0 | 0 | 0 | 0 | 0 | −21.307 | 0 | 0 | 0 | 0 | −17 |
| SER B 112 | 0 | 0 | 0 | 0 | 0 | 0 | 0 | 0 | 0 | 0 | 0 | 0 |
| SER B 113 | 0 | 0 | 0 | 0 | 0 | 0 | 0 | 0 | 0 | 0 | 0 | 0 |
| PHE B 114 | 0 | 0 | 0 | 0 | 0 | 0 | 0 | 0 | 0 | 0 | 0 | 0 |
| VAL B 115 | 0 | 0 | 0 | 0 | 0 | 0 | 0 | 0 | 0 | 0 | 0 | 0 |
| CYS B 116 | 0 | 0 | 0 | 0 | 0 | 0 | 0 | 0 | 0 | 0 | 0 | 0 |
| VAL B 117 | 0 | 0 | 0 | 0 | 0 | 0 | 0 | 0 | 0 | 0 | 0 | 0 |
| LEU B 118 | 0 | 0 | 0 | 0 | 0 | 0 | 0 | 0 | 0 | 0 | 0 | 0 |
| LEU B 119 | 0 | 0 | 0 | 0 | 0 | 0 | 0 | 0 | 0 | 0 | 0 | 0 |
| SER B 120 | −26.098 | −16.530 | 0 | −21.593 | −27.171 | −26.233 | −35.307 | −4.330 | −29.472 | −19.976 | −30.528 | −21 |
| HIP B 121 | −31.098 | −25.530 | 0 | −27.593 | −29.171 | −26.233 | −35.307 | −6.330 | −29.472 | −19.976 | −35.528 | −23 |
| GLY B 122 | −31.098 | −25.530 | 0 | −17.593 | −29.171 | −22.233 | −31.307 | −4.330 | −16.472 | −19.976 | −35.528 | −21 |
| GLU B 123 | 0 | −25.530 | 0 | 0 | −29.171 | 0 | 0 | 0 | 0 | 0 | 0 | 0 |
| GLU B 124 | 0 | 0 | 0 | 0 | 0 | 0 | 0 | 0 | −13.472 | 0 | 0 | 0 |
| GLY B 125 | 0 | 0 | 0 | 0 | 0 | 0 | 0 | 0 | 0 | 0 | 0 | 0 |
| ILE B 126 | 0 | 0 | 0 | 0 | 0 | 0 | 0 | 0 | 0 | 0 | 0 | 0 |
| ILE B 127 | 0 | 0 | 0 | 0 | 0 | 0 | 0 | 0 | 0 | 0 | 0 | 0 |
| PHE B 128 | 0 | −16.530 | 0 | 0 | 0 | 0 | 0 | 0 | 0 | 0 | 0 | 0 |
| GLY B 129 | 0 | 0 | 0 | 0 | 0 | 0 | 0 | 0 | 0 | 0 | 0 | 0 |
| THR B 130 | 0 | 0 | 0 | 0 | 0 | 0 | 0 | 0 | 0 | 0 | 0 | 0 |
| ASN B 131 | 0 | 0 | 0 | 0 | 0 | 0 | 0 | 0 | 0 | 0 | 0 | 0 |
| GLY B 132 | 0 | 0 | 0 | 0 | 0 | 0 | 0 | 0 | 0 | 0 | 0 | 0 |
| PRO B 133 | 0 | 0 | 0 | 0 | 0 | 0 | 0 | 0 | 0 | 0 | 0 | 0 |
| VAL B 134 | 0 | 0 | 0 | 0 | 0 | 0 | 0 | 0 | 0 | 0 | 0 | 0 |
| ASP B 135 | 0 | 0 | 0 | 0 | −20.171 | 0 | 0 | 0 | 0 | 0 | 0 | 0 |
| LEU B 136 | 0 | 0 | 0 | 0 | 0 | 0 | 0 | 0 | 0 | 0 | 0 | 0 |
| LYS B 137 | −18.098 | 0 | 0 | 0 | 0 | 0 | −24.307 | 0 | −9.472 | 0 | −20.528 | 0 |
| LYS B 138 | −24.098 | −19.530 | −5.808 | 0 | −25.171 | 0 | −30.307 | 0 | −16.472 | −13.976 | −23.528 | −16 |
| ILE B 139 | 0 | 0 | 0 | 0 | 0 | 0 | 0 | 0 | 0 | 0 | 0 | 0 |
| THR B 140 | 0 | 0 | 0 | 0 | 0 | 0 | 0 | 0 | 0 | 0 | 0 | 0 |
| ASN B 141 | −18.098 | 0 | 0 | 0 | 0 | 0 | −24.307 | 0 | −10.472 | 0 | −20.528 | 0 |
| PHE B 142 | 0 | 0 | 0 | 0 | 0 | 0 | 0 | 0 | 0 | 0 | 0 | 0 |
| PHE B 143 | 0 | 0 | 0 | 0 | 0 | 0 | 0 | 0 | 0 | 0 | 0 | 0 |
| ARG B 144 | −26.098 | −16.530 | 0 | 0 | −25.171 | 0 | −30.307 | 0 | −10.472 | −10.976 | −25.528 | 0 |
| GLY B 145 | 0 | 0 | 0 | 0 | 0 | 0 | 0 | 0 | 0 | 0 | 0 | 0 |
| ASP B 146 | 0 | 0 | 0 | 0 | 0 | 0 | 0 | 0 | 0 | 0 | 0 | 0 |
| ARG B 147 | 0 | 0 | 0 | 0 | 0 | 0 | 0 | 0 | 0 | 0 | 0 | 0 |
| CYS B 148 | 0 | 0 | 0 | 0 | 0 | 0 | 0 | 0 | 0 | 0 | 0 | 0 |
| ARG B 149 | 0 | 0 | 0 | 0 | 0 | 0 | 0 | 0 | 0 | 0 | 0 | 0 |
| SER B 150 | 0 | 0 | 0 | 0 | 0 | 0 | 0 | 0 | 0 | 0 | 0 | 0 |
| LEU B 151 | 0 | 0 | 0 | 0 | 0 | 0 | 0 | 0 | 0 | 0 | 0 | 0 |
| THR B 152 | 0 | 0 | 0 | 0 | 0 | 0 | 0 | 0 | 0 | 0 | 0 | −20 |
| GLY B 153 | −23.098 | −15.530 | −7.808 | 0 | −20.171 | 0 | −27.307 | −4.330 | −11.472 | −15.976 | 0 | −20 |
| LYS B 154 | 0 | 0 | 0 | 0 | 0 | 0 | 0 | 0 | 0 | 0 | 0 | 0 |
| PRO B 155 | 0 | 0 | 0 | 0 | 0 | 0 | 0 | 0 | 0 | 0 | 0 | 0 |
| LYS B 156 | 0 | 0 | 0 | 0 | 0 | 0 | 0 | 0 | 0 | 0 | 0 | −20 |
| LEU B 157 | 0 | 0 | 0 | 0 | 0 | 0 | 0 | 0 | 0 | 0 | 0 | 0 |
| PHE B 158 | 0 | 0 | 0 | 0 | 0 | 0 | 0 | 0 | 0 | 0 | 0 | 0 |
| ILE B 159 | 0 | 0 | 0 | 0 | 0 | 0 | 0 | 0 | 0 | 0 | 0 | 0 |
| ILE B 160 | 0 | 0 | 0 | 0 | 0 | 0 | 0 | 0 | 0 | 0 | 0 | 0 |
| GLN B 161 | −27.098 | −16.530 | 0 | 0 | −29.171 | −18.233 | −19.307 | −4.330 | −17.472 | −19.976 | −26.528 | 0 |
| ALA B 162 | −31.098 | −25.530 | 0 | −21.593 | −29.171 | −26.233 | −35.307 | −4.330 | −17.472 | −19.976 | −34.528 | −21 |
| CYM B 163 | −31.098 | −25.530 | 0 | −27.593 | −29.171 | −26.233 | −35.307 | −6.330 | −29.472 | −19.976 | −34.528 | −23 |
| ARG B 164 | 0 | 0 | 0 | 0 | 0 | 0 | 0 | 0 | −14.472 | 0 | 0 | 0 |
| GLY B 165 | 0 | 0 | 0 | 0 | 0 | 0 | 0 | 0 | 0 | 0 | 0 | 0 |
| THR B 166 | 0 | 0 | 0 | 0 | 0 | 0 | 0 | 0 | 0 | 0 | 0 | 0 |
| GLU B 167 | −24.098 | −13.530 | 0 | −13.593 | −16.171 | −15.233 | −25.307 | 0 | 0 | −9.976 | −23.528 | −21 |
| LEU B 168 | −24.098 | 0 | −5.808 | 0 | −16.171 | −15.233 | −21.307 | 0 | 0 | −9.976 | 0 | 0 |
| ASP B 169 | 0 | 0 | 0 | 0 | 0 | 0 | 0 | 0 | 0 | 0 | 0 | 0 |
| CYS B 170 | 0 | 0 | 0 | 0 | 0 | 0 | 0 | 0 | 0 | 0 | 0 | 0 |
| GLY B 171 | 0 | 0 | 0 | 0 | 0 | 0 | 0 | 0 | 0 | 0 | 0 | 0 |
| ILE B 172 | 0 | 0 | 0 | 0 | 0 | 0 | 0 | 0 | 0 | 0 | 0 | 0 |
| GLU B 173 | 0 | 0 | 0 | 0 | 0 | 0 | −19.307 | 0 | 0 | 0 | −22.528 | 0 |
| NME B 999 | 0 | 0 | 0 | 0 | 0 | 0 | 0 | 0 | 0 | 0 | 0 | 0 |
| ACE F 0 | 0 | 0 | 0 | 0 | 0 | 0 | 0 | 0 | 0 | 0 | 0 | 0 |
| HID F 185 | 0 | −14.530 | −7.808 | 0 | −20.171 | 0 | −27.307 | −4.330 | −11.472 | −15.976 | 0 | 0 |
| LYS F 186 | 0 | 0 | 0 | −16.593 | 0 | 0 | 0 | 0 | 0 | 0 | 0 | 0 |
| ILE F 187 | −23.098 | −14.530 | 0 | 0 | −20.171 | 0 | −27.307 | −4.330 | −11.472 | −15.976 | 0 | −20 |
| PRO F 188 | 0 | 0 | 0 | 0 | 0 | 0 | 0 | 0 | 0 | 0 | 0 | 0 |
| VAL F 189 | 0 | 0 | 0 | 0 | 0 | 0 | 0 | 0 | 0 | 0 | 0 | 0 |
| ASP F 190 | 0 | 0 | 0 | 0 | 0 | 0 | 0 | 0 | 0 | 0 | 0 | 0 |
| ALA F 191 | 0 | 0 | 0 | 0 | 0 | 0 | 0 | 0 | 0 | 0 | 0 | −20 |
| ASP F 192 | −23.098 | −14.530 | 0 | 0 | −20.171 | 0 | −27.307 | 0 | −11.472 | −15.976 | 0 | −20 |
| PHE F 193 | 0 | 0 | 0 | 0 | 0 | 0 | 0 | 0 | 0 | 0 | 0 | 0 |
| LEU F 194 | 0 | 0 | 0 | 0 | 0 | 0 | 0 | 0 | 0 | 0 | 0 | 0 |
| TYR F 195 | 0 | 0 | 0 | 0 | 0 | 0 | 0 | 0 | 0 | 0 | 0 | 0 |
| ALA F 196 | 0 | 0 | 0 | 0 | 0 | 0 | 0 | 0 | 0 | 0 | 0 | 0 |
| TYR F 197 | 0 | 0 | 0 | 0 | 0 | 0 | 0 | 0 | 0 | 0 | 0 | 0 |
| SER F 198 | 0 | 0 | 0 | 0 | 0 | 0 | 0 | 0 | 0 | 0 | 0 | 0 |
| THR F 199 | 0 | 0 | 0 | 0 | 0 | 0 | 0 | 0 | 0 | 0 | 0 | 0 |
| ALA F 200 | 0 | 0 | 0 | 0 | 0 | 0 | 0 | 0 | 0 | 0 | 0 | 0 |
| PRO F 201 | 0 | 0 | −7.808 | −13.593 | 0 | −11.233 | 0 | −4.330 | −12.472 | 0 | 0 | −21 |
| GLY F 202 | 0 | 0 | 0 | −13.593 | 0 | 0 | 0 | 0 | 0 | 0 | 0 | 0 |
| TYR F 203 | −24.098 | 0 | −7.808 | 0 | 0 | −15.233 | 0 | 0 | 0 | 0 | −18.528 | −21 |
| TYR F 204 | 0 | 0 | 0 | 0 | −29.171 | −11.233 | 0 | −4.330 | −17.472 | −15.976 | 0 | 0 |
| SER F 205 | −31.098 | −25.530 | 0 | −21.593 | −29.171 | −26.233 | −35.307 | −4.330 | −29.472 | −19.976 | −34.528 | −21 |
| TRP F 206 | 0 | 0 | 0 | 0 | 0 | 0 | 0 | −4.330 | −17.472 | 0 | 0 | 0 |
| ARG F 207 | −31.098 | −25.530 | −5.808 | −21.593 | −29.171 | −26.233 | −35.307 | −4.330 | −29.472 | −19.976 | −34.528 | 0 |
| ASN F 208 | 0 | 0 | 0 | 0 | 0 | 0 | 0 | 0 | −10.472 | 0 | 0 | 0 |
| SER F 209 | 0 | 0 | 0 | 0 | 0 | 0 | 0 | 0 | 0 | 0 | 0 | 0 |
| LYS F 210 | 0 | 0 | 0 | 0 | 0 | 0 | 0 | 0 | 0 | 0 | 0 | 0 |
| ASP F 211 | 0 | 0 | 0 | 0 | 0 | 0 | 0 | 0 | 0 | 0 | 0 | 0 |
| GLY F 212 | 0 | 0 | 0 | 0 | 0 | 0 | 0 | 0 | 0 | 0 | 0 | 0 |
| SER F 213 | 0 | 0 | 0 | 0 | 0 | 0 | 0 | 0 | 0 | 0 | 0 | 0 |
| TRP F 214 | 0 | 0 | 0 | 0 | 0 | 0 | 0 | 0 | −10.472 | 0 | 0 | 0 |
| PHE F 215 | 0 | 0 | 0 | 0 | 0 | 0 | 0 | 0 | 0 | 0 | 0 | 0 |
| ILE F 216 | 0 | 0 | 0 | 0 | 0 | 0 | 0 | 0 | 0 | 0 | 0 | 0 |
| GLN F 217 | 0 | 0 | 0 | 0 | 0 | 0 | 0 | 0 | 0 | 0 | 0 | 0 |
| SER F 218 | 0 | 0 | 0 | 0 | 0 | 0 | 0 | 0 | 0 | 0 | 0 | 0 |
| LEU F 219 | 0 | 0 | 0 | 0 | 0 | 0 | 0 | 0 | 0 | 0 | 0 | 0 |
| CYS F 220 | 0 | 0 | 0 | 0 | 0 | 0 | 0 | 0 | 0 | 0 | 0 | 0 |
| ALA F 221 | 0 | 0 | 0 | 0 | 0 | 0 | 0 | 0 | 0 | 0 | 0 | 0 |
| MET F 222 | 0 | 0 | −6.808 | 0 | 0 | 0 | 0 | 0 | −13.472 | −13.976 | 0 | 0 |
| LEU F 223 | 0 | 0 | 0 | 0 | 0 | 0 | 0 | 0 | 0 | 0 | 0 | 0 |
| LYS F 224 | 0 | 0 | 0 | 0 | 0 | 0 | 0 | 0 | 0 | 0 | 0 | 0 |
| GLN F 225 | 0 | 0 | 0 | 0 | −16.171 | 0 | 0 | 0 | 0 | 0 | 0 | 0 |
| TYR F 226 | 0 | 0 | 0 | 0 | 0 | 0 | 0 | 0 | 0 | 0 | 0 | 0 |
| ALA F 227 | 0 | 0 | 0 | 0 | 0 | 0 | 0 | 0 | 0 | 0 | 0 | 0 |
| ASP F 228 | 0 | 0 | 0 | 0 | −16.171 | 0 | 0 | 0 | 0 | 0 | −17.528 | 0 |
| LYS F 229 | 0 | 0 | 0 | 0 | −16.171 | 0 | 0 | 0 | 0 | 0 | −17.528 | 0 |
| LEU F 230 | −25.098 | −18.530 | 0 | −19.593 | −19.171 | 0 | 0 | 0 | −12.472 | 0 | −22.528 | 0 |
| GLU F 231 | 0 | 0 | 0 | 0 | 0 | 0 | 0 | 0 | 0 | 0 | 0 | 0 |
| PHE F 232 | 0 | 0 | 0 | 0 | 0 | 0 | 0 | 0 | 0 | 0 | 0 | 0 |
| MET F 233 | 0 | 0 | 0 | 0 | 0 | 0 | 0 | 0 | 0 | 0 | 0 | 0 |
| HIE F 234 | 0 | 0 | 0 | 0 | 0 | 0 | 0 | 0 | 0 | 0 | 0 | 0 |
| ILE F 235 | 0 | 0 | 0 | 0 | 0 | 0 | 0 | 0 | 0 | 0 | 0 | 0 |
| LEU F 236 | 0 | 0 | 0 | 0 | 0 | 0 | 0 | 0 | 0 | 0 | 0 | 0 |
| THR F 237 | 0 | 0 | 0 | 0 | 0 | 0 | 0 | 0 | 0 | 0 | 0 | 0 |
| ARG F 238 | −25.098 | −18.530 | 0 | −19.593 | −23.171 | −22.233 | −27.307 | 0 | −13.472 | −12.976 | −22.528 | −21 |
| VAL F 239 | 0 | 0 | 0 | 0 | 0 | 0 | 0 | 0 | 0 | 0 | 0 | 0 |
| ASN F 240 | 0 | 0 | 0 | 0 | 0 | 0 | 0 | 0 | 0 | 0 | 0 | 0 |
| ARG F 241 | −24.098 | −15.530 | −8.808 | 0 | −20.171 | 0 | −26.307 | 0 | −13.472 | −13.976 | −23.528 | 0 |
| LYS F 242 | −24.098 | −13.530 | −5.808 | 0 | 0 | 0 | −26.307 | 0 | −13.472 | −13.976 | −20.528 | 0 |
| VAL F 243 | 0 | 0 | 0 | 0 | 0 | 0 | 0 | 0 | 0 | 0 | 0 | 0 |
| ALA F 244 | 0 | 0 | 0 | 0 | 0 | 0 | 0 | 0 | 0 | 0 | 0 | 0 |
| THR F 245 | −24.098 | −15.530 | −6.808 | −19.593 | −18.171 | 0 | −19.307 | 0 | −13.472 | −9.976 | −23.528 | 0 |
| GLU F 246 | −24.098 | −15.530 | 0 | 0 | −20.171 | 0 | −26.307 | 0 | −13.472 | −13.976 | −23.528 | 0 |
| PHE F 247 | 0 | −15.530 | 0 | 0 | 0 | 0 | 0 | 0 | 0 | 0 | 0 | 0 |
| GLU F 248 | 0 | −15.530 | 0 | −19.593 | −18.171 | −12.233 | 0 | 0 | −10.472 | 0 | −18.528 | −16 |
| SER F 249 | 0 | 0 | 0 | 0 | 0 | 0 | 0 | 0 | −10.472 | 0 | 0 | 0 |
| PHE F 250 | 0 | 0 | 0 | 0 | 0 | 0 | 0 | 0 | −10.472 | 0 | 0 | 0 |
| SER F 251 | 0 | 0 | 0 | 0 | 0 | 0 | 0 | 0 | 0 | 0 | 0 | 0 |
| PHE F 252 | 0 | 0 | 0 | 0 | 0 | 0 | 0 | 0 | 0 | 0 | 0 | 0 |
| ASP F 253 | 0 | 0 | 0 | 0 | 0 | 0 | 0 | 0 | 0 | 0 | 0 | 0 |
| ALA F 254 | 0 | 0 | 0 | 0 | 0 | 0 | 0 | 0 | 0 | 0 | 0 | 0 |
| THR F 255 | 0 | 0 | 0 | 0 | 0 | 0 | 0 | 0 | 0 | 0 | 0 | 0 |
| PHE F 256 | 0 | 0 | 0 | 0 | 0 | 0 | 0 | 0 | 0 | 0 | 0 | 0 |
| HIE F 257 | 0 | 0 | 0 | 0 | 0 | 0 | 0 | 0 | 0 | 0 | 0 | 0 |
| ALA F 258 | 0 | 0 | 0 | −12.593 | 0 | −12.233 | 0 | 0 | 0 | 0 | 0 | 0 |
| LYS F 259 | 0 | 0 | 0 | 0 | 0 | 0 | 0 | 0 | 0 | 0 | 0 | 0 |
| LYS F 260 | 0 | −15.530 | 0 | −19.593 | −18.171 | 0 | 0 | 0 | 0 | 0 | −18.528 | −16 |
| GLN F 261 | 0 | 0 | 0 | 0 | 0 | 0 | 0 | 0 | 0 | 0 | 0 | 0 |
| ILE F 262 | 0 | 0 | 0 | 0 | 0 | 0 | 0 | 0 | 0 | 0 | 0 | 0 |
| PRO F 263 | 0 | 0 | 0 | 0 | 0 | 0 | 0 | 0 | 0 | 0 | 0 | 0 |
| CYS F 264 | 0 | 0 | 0 | 0 | 0 | 0 | 0 | 0 | 0 | 0 | 0 | 0 |
| ILE F 265 | 0 | 0 | 0 | 0 | 0 | 0 | 0 | 0 | 0 | 0 | 0 | 0 |
| VAL F 266 | 0 | 0 | 0 | 0 | 0 | 0 | 0 | 0 | 0 | 0 | 0 | 0 |
| SER F 267 | 0 | 0 | 0 | 0 | 0 | 0 | 0 | 0 | 0 | 0 | 0 | 0 |
| MET F 268 | 0 | 0 | 0 | 0 | 0 | 0 | 0 | 0 | 0 | 0 | 0 | 0 |
| LEU F 269 | 0 | 0 | 0 | 0 | 0 | 0 | 0 | 0 | 0 | 0 | 0 | 0 |
| THR F 270 | −23.098 | −14.530 | 0 | 0 | −20.171 | 0 | −27.307 | −4.330 | −11.472 | −15.976 | 0 | 0 |
| LYS F 271 | −23.098 | −15.530 | −7.808 | 0 | −20.171 | 0 | −27.307 | −4.330 | −11.472 | −15.976 | 0 | 0 |
| GLU F 272 | −18.098 | −13.530 | 0 | −16.593 | −17.171 | −15.233 | −21.307 | 0 | 0 | 0 | −20.528 | −26 |
| LEU F 273 | 0 | 0 | 0 | 0 | 0 | 0 | 0 | 0 | 0 | 0 | 0 | 0 |
| TYR F 274 | 0 | −13.530 | 0 | 0 | 0 | 0 | 0 | 0 | 0 | 0 | 0 | 0 |
| PHE F 275 | 0 | 0 | 0 | 0 | 0 | 0 | 0 | 0 | 0 | 0 | 0 | 0 |
| TYR F 276 | 0 | 0 | 0 | 0 | 0 | 0 | 0 | 0 | 0 | 0 | 0 | 0 |
| HID F 277 | 0 | 0 | 0 | 0 | 0 | 0 | 0 | 0 | 0 | 0 | 0 | 0 |
| NME F 999 | 0 | 0 | 0 | 0 | 0 | 0 | 0 | 0 | 0 | 0 | 0 | 0 |

### Example 2

The following data in Table 2 was generated from a simulation conducted according to the methods of the present invention on the protein Caspase-8. Amino acids are listed on the left hand side, while the fragments are listed at the top. The binding affinities associated with the fragment-residue pairs are listed.

TABLE 2
Fragment Binding Affinity for Caspase-8

| | | | | | | | | | | tetra- | | |
|---|---|---|---|---|---|---|---|---|---|---|---|---|
| | acet- | | | carbox- | dimethyl | | | iso- | pyrimi- | hydro- | | |
| | amide | acetone | benzene | ylic acid | sulfoxide | ethanol | imidazole | butane | dine | furan | urea | H₂O |
| ACE A 0 | −27.098 | −8.530 | 0 | 0 | −11.171 | 0 | −14.307 | 0 | 0 | −6.976 | −22.528 | −17 |
| ASP A 223 | −27.098 | −20.530 | −3.808 | −12.593 | −22.171 | −13.233 | −30.307 | 0 | −14.472 | −11.976 | −22.528 | −17 |
| LYS A 224 | −27.098 | −20.530 | −3.808 | −12.593 | −22.171 | −14.233 | −30.307 | 0 | −14.472 | −11.976 | −22.528 | −16 |
| VAL A 225 | −13.098 | −11.530 | −4.808 | −12.593 | −15.171 | −10.233 | 0 | −2.330 | −9.472 | −9.976 | −19.528 | 0 |
| TYR A 226 | −26.098 | −21.530 | −4.808 | −10.593 | −26.171 | −16.233 | −20.307 | −2.330 | −11.472 | −11.976 | −20.528 | −11 |
| GLN A 227 | −26.098 | −21.530 | −4.808 | −10.593 | −26.171 | −11.233 | −20.307 | −2.330 | −11.472 | −11.976 | −20.528 | −11 |
| MET A 228 | −13.098 | −12.530 | 0 | −10.593 | −18.171 | 0 | −13.307 | 0 | −9.472 | −8.976 | −22.528 | 0 |
| LYS A 229 | −22.098 | −12.530 | −4.808 | −13.593 | −18.171 | −15.233 | −25.307 | −2.330 | −11.472 | −7.976 | −30.528 | 0 |
| SER A 230 | 0 | 0 | 0 | 0 | 0 | 0 | 0 | 0 | 0 | 0 | 0 | 0 |
| LYS A 231 | 0 | 0 | 0 | −13.593 | 0 | −15.233 | 0 | 0 | −7.472 | 0 | 0 | −12 |
| PRO A 232 | 0 | 0 | 0 | 0 | 0 | 0 | 0 | 0 | 0 | 0 | 0 | 0 |
| ARG A 233 | 0 | 0 | 0 | −13.593 | 0 | −15.233 | 0 | 0 | 0 | 0 | 0 | −12 |
| GLY A 234 | 0 | 0 | 0 | 0 | 0 | 0 | 0 | 0 | 0 | 0 | 0 | 0 |
| TYR A 235 | 0 | 0 | 0 | −8.593 | 0 | 0 | 0 | 0 | 0 | 0 | 0 | 0 |
| CYS A 236 | 0 | 0 | 0 | 0 | 0 | 0 | 0 | 0 | 0 | 0 | 0 | 0 |
| LEU A 237 | 0 | 0 | 0 | 0 | 0 | 0 | 0 | 0 | 0 | 0 | 0 | 0 |
| ILE A 238 | 0 | 0 | 0 | 0 | 0 | 0 | 0 | 0 | 0 | 0 | 0 | 0 |
| ILE A 239 | 0 | 0 | 0 | 0 | 0 | 0 | 0 | 0 | 0 | 0 | 0 | 0 |
| ASN A 240 | 0 | 0 | 0 | 0 | 0 | 0 | 0 | 0 | 0 | 0 | 0 | 0 |
| ASN A 241 | 0 | 0 | 0 | 0 | 0 | 0 | 0 | 0 | 0 | 0 | 0 | 0 |
| HID A 242 | 0 | −11.530 | 0 | 0 | 0 | 0 | −14.307 | 0 | 0 | 0 | 0 | 0 |
| ASN A 243 | 0 | 0 | 0 | 0 | 0 | −9.233 | 0 | 0 | 0 | 0 | 0 | 0 |
| PHE A 244 | 0 | 0 | 0 | 0 | 0 | 0 | 0 | 0 | 0 | 0 | 0 | 0 |
| ALA A 245 | 0 | 0 | −3.808 | 0 | 0 | 0 | 0 | 0 | 0 | 0 | 0 | 0 |
| LYS A 246 | −15.098 | −11.530 | −4.808 | −8.593 | −15.171 | −9.233 | −17.307 | 0 | −15.472 | −6.976 | −12.528 | −11 |
| ALA A 247 | 0 | 0 | 0 | 0 | 0 | 0 | 0 | 0 | 0 | 0 | 0 | 0 |
| ARG A 248 | 0 | 0 | 0 | 0 | 0 | 0 | 0 | 0 | 0 | 0 | 0 | 0 |
| GLU A 249 | 0 | 0 | 0 | 0 | 0 | 0 | 0 | 0 | 0 | 0 | 0 | 0 |
| LYS A 250 | −28.098 | −12.530 | −3.808 | −15.593 | −11.171 | −13.233 | −23.307 | 0 | −15.472 | −8.976 | −32.528 | −14 |
| VAL A 251 | 0 | 0 | 0 | 0 | 0 | 0 | 0 | 0 | 0 | 0 | 0 | −11 |
| PRO A 252 | 0 | 0 | 0 | 0 | 0 | 0 | 0 | 0 | 0 | 0 | 0 | 0 |
| LYS A 253 | −14.098 | −8.530 | 0 | −11.593 | −10.171 | −12.233 | −14.307 | −2.330 | −9.472 | 0 | −17.528 | −14 |
| LEU A 254 | 0 | 0 | 0 | 0 | 0 | 0 | 0 | −2.330 | 0 | 0 | 0 | 0 |
| HID A 255 | 0 | 0 | 0 | 0 | 0 | 0 | 0 | 0 | 0 | 0 | 0 | 0 |
| SER A 256 | 0 | 0 | 0 | 0 | 0 | 0 | 0 | 0 | 0 | 0 | 0 | 0 |
| ILE A 257 | 0 | 0 | −3.808 | 0 | 0 | 0 | 0 | −3.330 | 0 | 0 | 0 | 0 |
| ARG A 258 | 0 | 0 | −8.808 | −7.593 | 0 | −12.233 | −15.307 | −2.330 | −11.472 | −6.976 | 0 | 0 |
| ASP A 259 | −14.098 | 0 | −8.808 | −7.593 | 0 | −12.233 | −15.307 | 0 | −11.472 | 0 | −12.528 | 0 |
| ARG A 260 | −30.098 | −25.530 | −8.808 | −22.593 | −28.171 | −18.233 | −32.307 | 0 | 0 | −16.976 | −31.528 | −10 |
| ASN A 261 | −14.098 | −11.530 | 0 | −7.593 | 0 | −12.233 | −15.307 | 0 | −11.472 | 0 | −12.528 | 0 |
| GLY A 262 | 0 | −11.530 | 0 | 0 | 0 | 0 | 0 | 0 | 0 | 0 | 0 | 0 |
| THR A 263 | 0 | −11.530 | 0 | 0 | 0 | 0 | 0 | 0 | 0 | 0 | 0 | 0 |
| HIE A 264 | 0 | −11.530 | 0 | 0 | 0 | 0 | −14.307 | 0 | 0 | 0 | 0 | 0 |
| LEU A 265 | 0 | 0 | 0 | 0 | 0 | 0 | 0 | 0 | 0 | 0 | 0 | 0 |
| ASP A 266 | 0 | 0 | 0 | 0 | 0 | 0 | 0 | 0 | 0 | 0 | 0 | 0 |
| ALA A 267 | 0 | 0 | 0 | 0 | 0 | 0 | 0 | 0 | 0 | 0 | 0 | 0 |
| GLY A 268 | 0 | 0 | 0 | 0 | 0 | 0 | 0 | 0 | 0 | 0 | 0 | 0 |
| ALA A 269 | 0 | 0 | 0 | 0 | 0 | 0 | 0 | −2.330 | 0 | 0 | 0 | 0 |
| LEU A 270 | 0 | 0 | 0 | 0 | 0 | 0 | 0 | 0 | 0 | 0 | 0 | 0 |
| THR A 271 | 0 | 0 | 0 | 0 | 0 | 0 | 0 | 0 | 0 | 0 | 0 | 0 |
| THR A 272 | 0 | 0 | 0 | 0 | 0 | 0 | 0 | 0 | 0 | 0 | 0 | 0 |
| THR A 273 | 0 | 0 | 0 | 0 | 0 | 0 | 0 | 0 | 0 | 0 | 0 | 0 |
| PHE A 274 | 0 | 0 | 0 | 0 | 0 | 0 | 0 | 0 | 0 | 0 | 0 | 0 |
| GLU A 275 | 0 | 0 | 0 | 0 | 0 | 0 | 0 | 0 | 0 | 0 | 0 | 0 |
| GLU A 276 | 0 | 0 | 0 | 0 | 0 | 0 | 0 | 0 | 0 | 0 | 0 | 0 |
| LEU A 277 | 0 | 0 | 0 | 0 | 0 | 0 | 0 | 0 | 0 | 0 | 0 | 0 |
| HIE A 278 | 0 | 0 | 0 | 0 | 0 | 0 | 0 | 0 | 0 | 0 | 0 | 0 |
| PHE A 279 | 0 | 0 | 0 | 0 | 0 | 0 | 0 | 0 | 0 | 0 | 0 | 0 |
| GLU A 280 | 0 | 0 | 0 | −9.593 | 0 | 0 | 0 | 0 | 0 | 0 | 0 | 0 |
| ILE A 281 | 0 | 0 | 0 | 0 | 0 | 0 | 0 | 0 | 0 | 0 | 0 | 0 |
| LYS A 282 | 0 | 0 | 0 | −9.593 | 0 | 0 | 0 | 0 | 0 | 0 | 0 | 0 |
| PRO A 283 | 0 | 0 | 0 | 0 | 0 | 0 | 0 | 0 | 0 | 0 | 0 | 0 |
| HID A 284 | 0 | 0 | 0 | 0 | 0 | 0 | 0 | 0 | 0 | 0 | 0 | 0 |
| ASP A 285 | 0 | 0 | 0 | 0 | 0 | 0 | −14.307 | −2.330 | 0 | 0 | 0 | 0 |
| ASP A 286 | −13.098 | −11.530 | −3.808 | −8.593 | −14.171 | −9.233 | −16.307 | 0 | −7.472 | 0 | −12.528 | −10 |
| CYS A 287 | 0 | 0 | 0 | 0 | 0 | 0 | 0 | 0 | 0 | 0 | 0 | 0 |
| THR A 288 | −15.098 | −11.530 | 0 | −15.593 | −12.171 | 0 | −13.307 | 0 | −15.472 | 0 | 0 | 0 |
| VAL A 289 | 0 | 0 | 0 | 0 | 0 | 0 | 0 | 0 | 0 | 0 | 0 | 0 |
| GLU A 290 | −28.098 | −12.530 | 0 | −15.593 | −11.171 | −13.233 | −23.307 | 0 | 0 | 0 | −32.528 | −10 |
| GLN A 291 | −15.098 | −11.530 | 0 | −8.593 | −14.171 | −9.233 | −18.307 | 0 | 0 | −6.976 | −12.528 | 0 |
| ILE A 292 | 0 | 0 | 0 | 0 | 0 | 0 | 0 | 0 | 0 | 0 | 0 | 0 |
| TYR A 293 | 0 | 0 | 0 | 0 | 0 | 0 | 0 | 0 | 0 | 0 | 0 | 0 |
| GLU A 294 | −15.098 | −9.530 | 0 | 0 | −13.171 | 0 | −21.307 | 0 | 0 | −6.976 | −15.528 | 0 |
| ILE A 295 | 0 | 0 | 0 | 0 | 0 | 0 | 0 | 0 | 0 | 0 | 0 | 0 |
| LEU A 296 | 0 | 0 | 0 | 0 | 0 | 0 | 0 | 0 | 0 | 0 | 0 | 0 |
| LYS A 297 | −15.098 | −9.530 | −3.808 | 0 | −13.171 | 0 | −21.307 | 0 | 0 | −6.976 | −15.528 | 0 |
| ILE A 298 | 0 | 0 | 0 | 0 | 0 | 0 | −13.307 | 0 | 0 | −6.976 | −15.528 | 0 |
| TYR A 299 | 0 | 0 | 0 | 0 | 0 | 0 | 0 | 0 | 0 | 0 | 0 | 0 |
| GLN A 300 | −13.098 | −11.530 | 0 | −11.593 | −12.171 | 0 | −13.307 | 0 | −8.472 | −8.976 | 0 | 0 |
| LEU A 301 | −13.098 | −11.530 | 0 | −11.593 | −12.171 | 0 | −13.307 | 0 | −8.472 | −8.976 | 0 | 0 |
| MET A 302 | −13.098 | −11.530 | 0 | −11.593 | −12.171 | 0 | −13.307 | 0 | −8.472 | −6.976 | 0 | 0 |
| ASP A 303 | −13.098 | −11.530 | 0 | 0 | −12.171 | 0 | 0 | 0 | 0 | 0 | 0 | 0 |
| HIE A 304 | 0 | 0 | 0 | 0 | 0 | 0 | 0 | 0 | 0 | 0 | 0 | 0 |
| SER A 305 | 0 | 0 | 0 | 0 | 0 | 0 | 0 | 0 | 0 | 0 | 0 | 0 |
| ASN A 306 | 0 | 0 | 0 | 0 | 0 | 0 | 0 | 0 | 0 | 0 | 0 | 0 |
| MET A 307 | 0 | 0 | 0 | 0 | 0 | 0 | 0 | 0 | 0 | 0 | 0 | 0 |
| ASP A 308 | 0 | 0 | 0 | 0 | 0 | 0 | −20.307 | 0 | −7.472 | 0 | −17.528 | −11 |
| CYS A 309 | 0 | 0 | 0 | 0 | 0 | 0 | 0 | 0 | 0 | 0 | 0 | 0 |
| PHE A 310 | 0 | 0 | 0 | 0 | 0 | 0 | 0 | 0 | 0 | 0 | 0 | 0 |
| ILE A 311 | 0 | 0 | 0 | 0 | 0 | 0 | 0 | 0 | 0 | 0 | 0 | 0 |
| CYS A 312 | 0 | 0 | 0 | 0 | 0 | 0 | 0 | 0 | 0 | 0 | 0 | 0 |
| CYS A 313 | 0 | 0 | 0 | 0 | 0 | 0 | 0 | 0 | 0 | 0 | 0 | 0 |
| ILE A 314 | 0 | 0 | 0 | 0 | 0 | 0 | 0 | 0 | 0 | 0 | 0 | 0 |
| LEU A 315 | 0 | 0 | 0 | 0 | 0 | 0 | 0 | 0 | 0 | 0 | 0 | 0 |
| SER A 316 | −27.098 | −25.530 | −8.808 | 0 | 0 | −18.233 | −25.307 | 0 | 0 | −16.976 | 0 | 0 |
| HIE A 317 | −26.098 | −25.530 | −8.808 | −22.593 | −28.171 | −18.233 | −32.307 | −3.330 | 0 | −16.976 | 0 | 0 |
| GLY A 318 | 0 | 0 | 0 | 0 | 0 | 0 | 0 | −2.330 | 0 | 0 | 0 | 0 |
| ASP A 319 | −14.098 | −10.530 | −3.808 | −11.593 | −15.171 | −11.233 | −20.307 | 0 | −9.472 | −7.976 | −17.528 | −14 |
| LYS A 320 | −23.098 | −15.530 | −3.808 | −8.593 | −18.171 | −9.233 | −26.307 | 0 | −10.472 | −7.976 | −24.528 | 0 |
| GLY A 321 | 0 | 0 | 0 | 0 | 0 | 0 | 0 | −2.330 | 0 | 0 | 0 | 0 |
| ILE A 322 | 0 | 0 | 0 | −12.593 | 0 | 0 | 0 | 0 | 0 | 0 | 0 | 0 |
| ILE A 323 | 0 | 0 | 0 | 0 | 0 | 0 | 0 | 0 | 0 | 0 | 0 | 0 |
| TYR A 324 | 0 | 0 | 0 | 0 | 0 | 0 | 0 | −2.330 | 0 | 0 | 0 | 0 |
| GLY A 325 | 0 | 0 | 0 | 0 | 0 | 0 | 0 | 0 | 0 | 0 | 0 | 0 |
| THR A 326 | 0 | 0 | 0 | 0 | 0 | 0 | 0 | 0 | 0 | 0 | 0 | 0 |
| ASP A 327 | −28.098 | −12.530 | 0 | −15.593 | −11.171 | −13.233 | −23.307 | 0 | −15.472 | −8.976 | −32.528 | −14 |
| GLY A 328 | 0 | 0 | 0 | 0 | 0 | 0 | 0 | 0 | 0 | 0 | 0 | 0 |
| GLN A 329 | 0 | 0 | 0 | −7.593 | −10.171 | 0 | 0 | 0 | 0 | 0 | 0 | −11 |
| GLU A 330 | 0 | −8.530 | 0 | −11.593 | −10.171 | −12.233 | −14.307 | 0 | −9.472 | 0 | −17.528 | −11 |
| ALA A 331 | 0 | 0 | 0 | 0 | 0 | 0 | 0 | 0 | 0 | 0 | 0 | 0 |
| PRO A 332 | 0 | 0 | 0 | 0 | 0 | 0 | 0 | 0 | 0 | 0 | 0 | 0 |
| ILE A 333 | 0 | 0 | 0 | 0 | 0 | 0 | 0 | 0 | 0 | 0 | 0 | 0 |
| TYR A 334 | 0 | 0 | 0 | 0 | 0 | 0 | 0 | −2.330 | 0 | 0 | 0 | 0 |
| GLU A 335 | −14.098 | 0 | 0 | 0 | 0 | −7.233 | −17.307 | 0 | 0 | 0 | −16.528 | 0 |
| LEU A 336 | 0 | 0 | 0 | 0 | 0 | 0 | 0 | 0 | 0 | 0 | 0 | 0 |
| THR A 337 | 0 | 0 | 0 | 0 | 0 | 0 | 0 | 0 | 0 | 0 | 0 | 0 |
| SER A 338 | −14.098 | 0 | 0 | 0 | 0 | −7.233 | 0 | 0 | 0 | 0 | 0 | 0 |
| GLN A 339 | −14.098 | 0 | 0 | 0 | 0 | 0 | −17.307 | 0 | 0 | 0 | −16.528 | 0 |
| PHE A 340 | 0 | 0 | 0 | 0 | 0 | 0 | 0 | 0 | 0 | 0 | 0 | 0 |
| THR A 341 | 0 | 0 | 0 | 0 | 0 | −8.233 | 0 | 0 | 0 | 0 | 0 | 0 |
| GLY A 342 | −16.098 | 0 | 0 | 0 | 0 | −7.233 | 0 | −2.330 | 0 | 0 | −13.528 | 0 |
| LEU A 343 | 0 | 0 | 0 | 0 | 0 | 0 | 0 | −2.330 | 0 | 0 | 0 | 0 |
| LYS A 344 | −14.098 | −8.530 | −3.808 | 0 | −11.171 | −8.233 | −17.307 | 0 | 0 | 0 | −16.528 | 0 |
| CYS A 345 | −13.098 | −11.530 | 0 | −11.593 | −10.171 | 0 | 0 | 0 | 0 | −6.976 | 0 | 0 |
| PRO A 346 | −13.098 | −11.530 | 0 | −11.593 | −12.171 | 0 | −13.307 | 0 | −8.472 | −6.976 | 0 | 0 |
| SER A 347 | −13.098 | −11.530 | 0 | −11.593 | −12.171 | 0 | −13.307 | 0 | −8.472 | −6.976 | 0 | 0 |
| LEU A 348 | 0 | 0 | 0 | 0 | 0 | 0 | 0 | 0 | 0 | 0 | 0 | 0 |
| ALA A 349 | −16.098 | 0 | 0 | 0 | 0 | −7.233 | 0 | −3.330 | 0 | 0 | −13.528 | −14 |
| GLY A 350 | −26.098 | −21.530 | 0 | −11.593 | −26.171 | −16.233 | −20.307 | −2.330 | −10.472 | −12.976 | −20.528 | −15 |
| LYS A 351 | −26.098 | −21.530 | 0 | −11.593 | −26.171 | 0 | −20.307 | −2.330 | −9.472 | −9.976 | −17.528 | −11 |
| PRO A 352 | 0 | 0 | 0 | 0 | 0 | 0 | 0 | 0 | 0 | 0 | 0 | −11 |
| LYS A 353 | −16.098 | 0 | 0 | 0 | 0 | −7.233 | 0 | 0 | 0 | 0 | −13.528 | −14 |
| VAL A 354 | 0 | 0 | 0 | 0 | 0 | 0 | 0 | 0 | 0 | 0 | 0 | 0 |
| PHE A 355 | 0 | 0 | 0 | 0 | 0 | 0 | 0 | 0 | 0 | 0 | 0 | 0 |
| PHE A 356 | 0 | 0 | 0 | 0 | 0 | 0 | 0 | 0 | 0 | 0 | 0 | 0 |
| ILE A 357 | 0 | 0 | 0 | 0 | 0 | 0 | 0 | 0 | 0 | 0 | 0 | 0 |
| GLN A 358 | −30.098 | 0 | −8.808 | −22.593 | −28.171 | −18.233 | −32.307 | 0 | 0 | −16.976 | −30.528 | 0 |
| ALA A 359 | −30.098 | −25.530 | −8.808 | −7.593 | −28.171 | −18.233 | −25.307 | 0 | 0 | −16.976 | −22.528 | 0 |
| CYS A 360 | −30.098 | −25.530 | −8.808 | −22.593 | −28.171 | −18.233 | −32.307 | −3.330 | 0 | −16.976 | −31.528 | 0 |
| GLN A 361 | 0 | 0 | 0 | 0 | 0 | 0 | 0 | 0 | 0 | 0 | 0 | 0 |
| GLY A 362 | 0 | 0 | −3.808 | 0 | 0 | 0 | −21.307 | 0 | 0 | −6.976 | 0 | 0 |
| ASP A 363 | −23.098 | −15.530 | −3.808 | −8.593 | −18.171 | −9.233 | −26.307 | −3.330 | −8.472 | −7.976 | −24.528 | 0 |
| ASN A 364 | −13.098 | −8.530 | −4.808 | 0 | −11.171 | −7.233 | −21.307 | 0 | −8.472 | −9.976 | −11.528 | 0 |
| TYR A 365 | −13.098 | 0 | −3.808 | 0 | 0 | −7.233 | −14.307 | 0 | −7.472 | −6.976 | −11.528 | 0 |
| GLN A 366 | −13.098 | 0 | 0 | 0 | 0 | −7.233 | −15.307 | −3.330 | −10.472 | −6.976 | −11.528 | 0 |
| LYS A 367 | −13.098 | −8.530 | −4.808 | 0 | −10.171 | −7.233 | −15.307 | −2.330 | −10.472 | −6.976 | −11.528 | 0 |
| GLY A 368 | 0 | 0 | 0 | 0 | 0 | 0 | 0 | −3.330 | 0 | 0 | 0 | 0 |
| ILE A 369 | 0 | 0 | −4.808 | 0 | 0 | 0 | 0 | −3.330 | 0 | −6.976 | 0 | 0 |
| PRO A 370 | 0 | 0 | 0 | 0 | 0 | 0 | 0 | 0 | 0 | 0 | 0 | 0 |
| VAL A 371 | −13.098 | −10.530 | 0 | 0 | 0 | 0 | 0 | −2.330 | 0 | 0 | −11.528 | 0 |
| GLU A 372 | −13.098 | 0 | 0 | 0 | 0 | 0 | 0 | 0 | 0 | 0 | −11.528 | 0 |
| THR A 373 | −13.098 | 0 | 0 | 0 | 0 | 0 | 0 | 0 | 0 | 0 | 0 | 0 |
| ASP A 374 | −13.098 | 0 | 0 | 0 | 0 | 0 | 0 | 0 | 0 | 0 | −11.528 | 0 |
| NME A 999 | 0 | 0 | 0 | 0 | 0 | 0 | 0 | 0 | 0 | 0 | 0 | 0 |
| ACE B 0 | 0 | 0 | 0 | 0 | −10.171 | 0 | 0 | 0 | 0 | 0 | 0 | 0 |
| THR B 390 | 0 | 0 | 0 | 0 | 0 | 0 | 0 | 0 | 0 | 0 | 0 | 0 |
| ARG B 391 | 0 | 0 | 0 | 0 | −11.171 | 0 | 0 | 0 | 0 | −6.976 | 0 | 0 |
| TYR B 392 | 0 | 0 | 0 | 0 | 0 | 0 | 0 | −2.330 | 0 | 0 | 0 | 0 |
| ILE B 393 | −16.098 | 0 | 0 | 0 | −23.171 | −7.233 | 0 | −3.330 | 0 | −9.976 | −13.528 | 0 |
| PRO B 394 | −16.098 | 0 | 0 | 0 | 0 | −7.233 | 0 | 0 | 0 | 0 | −13.528 | 0 |
| ASP B 395 | −16.098 | 0 | 0 | 0 | 0 | −8.233 | 0 | −2.330 | 0 | 0 | −13.528 | 0 |
| GLU B 396 | 0 | 0 | 0 | 0 | 0 | 0 | 0 | 0 | 0 | 0 | 0 | 0 |
| ALA B 397 | −16.098 | 0 | 0 | 0 | 0 | −7.233 | 0 | 0 | 0 | 0 | −13.528 | −14 |
| ASP B 398 | −16.098 | 0 | 0 | 0 | 0 | −7.233 | 0 | 0 | 0 | 0 | −13.528 | −14 |
| PHE B 399 | 0 | 0 | 0 | 0 | 0 | 0 | 0 | −4.330 | 0 | 0 | 0 | 0 |
| LEU B 400 | 0 | 0 | 0 | 0 | 0 | 0 | 0 | 0 | 0 | 0 | 0 | 0 |
| LEU B 401 | 0 | 0 | −3.808 | 0 | 0 | 0 | 0 | −3.330 | 0 | 0 | 0 | 0 |
| GLY B 402 | 0 | 0 | 0 | 0 | 0 | 0 | 0 | 0 | 0 | 0 | 0 | 0 |
| MET B 403 | 0 | 0 | 0 | 0 | 0 | 0 | 0 | 0 | 0 | 0 | 0 | 0 |
| ALA B 404 | 0 | 0 | 0 | 0 | 0 | 0 | 0 | 0 | 0 | 0 | 0 | 0 |
| THR B 405 | 0 | 0 | 0 | 0 | 0 | 0 | 0 | 0 | 0 | 0 | 0 | 0 |
| VAL B 406 | 0 | 0 | 0 | 0 | 0 | 0 | 0 | 0 | 0 | 0 | 0 | 0 |
| ASN B 407 | 0 | 0 | 0 | 0 | 0 | 0 | 0 | 0 | 0 | 0 | 0 | 0 |
| ASN B 408 | 0 | 0 | 0 | 0 | 0 | 0 | −21.307 | 0 | 0 | 0 | 0 | 0 |
| CYS B 409 | −13.098 | 0 | 0 | 0 | 0 | 0 | 0 | 0 | 0 | 0 | −11.528 | 0 |
| VAL B 410 | 0 | 0 | 0 | 0 | 0 | 0 | 0 | −2.330 | 0 | 0 | 0 | 0 |
| SER B 411 | −30.098 | −25.530 | −8.808 | −22.593 | −28.171 | −18.233 | −32.307 | 0 | 0 | −16.976 | −31.528 | 0 |
| TYR B 412 | −30.098 | 0 | −8.808 | 0 | 0 | 0 | 0 | −3.330 | 0 | −7.976 | 0 | 0 |
| ARG B 413 | −30.098 | −25.530 | −8.808 | −22.593 | −28.171 | −18.233 | −32.307 | −3.330 | −11.472 | −16.976 | −31.528 | 0 |
| ASN B 414 | 0 | −9.530 | 0 | 0 | −10.171 | 0 | 0 | −3.330 | 0 | 0 | −13.528 | 0 |
| PRO B 415 | −14.098 | 0 | −8.808 | 0 | 0 | −9.233 | −15.307 | 0 | 0 | 0 | −12.528 | 0 |
| ALA B 416 | 0 | 0 | 0 | 0 | 0 | 0 | 0 | 0 | 0 | 0 | 0 | 0 |
| GLU B 417 | 0 | −9.530 | 0 | 0 | −15.171 | 0 | 0 | 0 | −15.472 | −8.976 | −13.528 | 0 |
| GLY B 418 | 0 | 0 | 0 | 0 | 0 | 0 | 0 | 0 | 0 | 0 | −13.528 | 0 |
| THR B 419 | 0 | 0 | −8.808 | 0 | −10.171 | 0 | 0 | 0 | 0 | 0 | 0 | 0 |
| TRP B 420 | 0 | −9.530 | −3.808 | 0 | −10.171 | 0 | 0 | −3.330 | −15.472 | −8.976 | −13.528 | 0 |
| TYR B 421 | 0 | 0 | 0 | 0 | 0 | 0 | 0 | 0 | 0 | 0 | 0 | 0 |
| ILE B 422 | 0 | 0 | 0 | 0 | 0 | 0 | 0 | 0 | 0 | 0 | 0 | 0 |
| GLN B 423 | −20.098 | −14.530 | −4.808 | 0 | −18.171 | 0 | −22.307 | −3.330 | −15.472 | −9.976 | −13.528 | 0 |
| SER B 424 | −21.098 | −19.530 | 0 | 0 | −18.171 | 0 | −22.307 | 0 | 0 | 0 | −20.528 | 0 |
| LEU B 425 | 0 | 0 | 0 | 0 | 0 | 0 | 0 | 0 | 0 | 0 | 0 | 0 |
| CYS B 426 | 0 | 0 | 0 | 0 | 0 | 0 | 0 | 0 | 0 | 0 | 0 | 0 |
| GLN B 427 | −21.098 | −19.530 | −3.808 | −9.593 | −18.171 | −12.233 | −22.307 | −2.330 | −12.472 | −8.976 | −22.528 | 0 |
| SER B 428 | 0 | 0 | 0 | 0 | 0 | 0 | 0 | 0 | 0 | 0 | 0 | 0 |
| LEU B 429 | 0 | 0 | 0 | 0 | 0 | 0 | 0 | 0 | 0 | 0 | 0 | 0 |
| ARG B 430 | 0 | 0 | 0 | 0 | 0 | 0 | 0 | −2.330 | 0 | 0 | 0 | 0 |
| GLU B 431 | −19.098 | −14.530 | 0 | 0 | −21.171 | −7.233 | −22.307 | 0 | −10.472 | −10.976 | −17.528 | −11 |
| ARG B 432 | 0 | 0 | 0 | 0 | −18.171 | −7.233 | 0 | 0 | 0 | 0 | −17.528 | −11 |
| CYS B 433 | 0 | 0 | 0 | 0 | 0 | 0 | 0 | 0 | 0 | 0 | 0 | 0 |
| PRO B 434 | 0 | 0 | 0 | 0 | 0 | 0 | 0 | 0 | 0 | 0 | 0 | 0 |
| ARG B 435 | −19.098 | −14.530 | 0 | 0 | −21.171 | 0 | −22.307 | 0 | −10.472 | −10.976 | −17.528 | 0 |
| GLY B 436 | 0 | 0 | 0 | 0 | −12.171 | 0 | 0 | 0 | 0 | 0 | 0 | 0 |
| ASP B 437 | −19.098 | −11.530 | 0 | −20.593 | −13.171 | 0 | 0 | 0 | −12.472 | −6.976 | −14.528 | 0 |
| ASP B 438 | −19.098 | −11.530 | 0 | −20.593 | −13.171 | −14.233 | −14.307 | 0 | −12.472 | −6.976 | −14.528 | −15 |
| ILE B 439 | 0 | 0 | 0 | 0 | 0 | 0 | 0 | 0 | 0 | 0 | 0 | 0 |
| LEU B 440 | 0 | 0 | 0 | 0 | 0 | 0 | 0 | 0 | 0 | 0 | 0 | 0 |
| THR B 441 | 0 | 0 | 0 | 0 | −13.171 | 0 | 0 | 0 | 0 | 0 | 0 | 0 |
| ILE B 442 | 0 | 0 | 0 | 0 | 0 | 0 | 0 | 0 | 0 | 0 | 0 | 0 |
| LEU B 443 | 0 | 0 | 0 | 0 | 0 | 0 | 0 | 0 | 0 | 0 | 0 | 0 |
| THR B 444 | 0 | 0 | 0 | 0 | 0 | 0 | 0 | 0 | 0 | 0 | 0 | 0 |
| GLU B 445 | 0 | 0 | 0 | 0 | 0 | −7.233 | 0 | 0 | 0 | 0 | −17.528 | −11 |
| VAL B 446 | 0 | 0 | 0 | 0 | 0 | 0 | 0 | 0 | 0 | 0 | 0 | 0 |
| ASN B 447 | 0 | 0 | 0 | 0 | 0 | 0 | 0 | 0 | 0 | 0 | 0 | 0 |
| TYR B 448 | −13.098 | 0 | 0 | 0 | 0 | −7.233 | 0 | −2.330 | 0 | 0 | −11.528 | 0 |
| GLU B 449 | −24.098 | −19.530 | 0 | −14.593 | −18.171 | −17.233 | −26.307 | 0 | −12.472 | −7.976 | −27.528 | −17 |
| VAL B 450 | 0 | 0 | 0 | 0 | 0 | 0 | 0 | 0 | 0 | 0 | 0 | 0 |
| SER B 451 | −13.098 | 0 | 0 | 0 | −10.171 | 0 | −13.307 | −3.330 | −10.472 | −6.976 | −11.528 | 0 |
| ASN B 452 | −24.098 | −10.530 | −4.808 | −11.593 | −10.171 | −9.233 | 0 | −2.330 | −10.472 | −7.976 | −27.528 | 0 |
| LYS B 453 | −24.098 | −19.530 | −5.808 | −14.593 | −18.171 | −17.233 | −26.307 | −3.330 | −15.472 | −11.976 | −27.528 | −17 |
| ASP B 454 | −16.098 | 0 | −4.808 | −12.593 | 0 | −7.233 | 0 | −2.330 | 0 | 0 | 0 | 0 |
| ASP B 455 | 0 | 0 | 0 | −7.593 | 0 | 0 | 0 | 0 | 0 | 0 | 0 | 0 |
| LYS B 456 | −16.098 | −9.530 | 0 | −12.593 | −11.171 | −7.233 | 0 | 0 | 0 | −6.976 | 0 | 0 |
| LYS B 457 | 0 | 0 | 0 | −7.593 | −11.171 | −7.233 | 0 | 0 | 0 | 0 | 0 | 0 |
| ASN B 458 | 0 | 0 | 0 | 0 | 0 | 0 | 0 | 0 | 0 | 0 | 0 | 0 |
| MET B 459 | 0 | 0 | 0 | 0 | 0 | 0 | 0 | 0 | 0 | 0 | 0 | 0 |
| GLY B 460 | 0 | 0 | 0 | 0 | 0 | 0 | 0 | 0 | 0 | 0 | 0 | 0 |
| LYS B 461 | −13.098 | −10.530 | −4.808 | −10.593 | −10.171 | −7.233 | −13.307 | −4.330 | −10.472 | −6.976 | −11.528 | 0 |
| GLN B 462 | 0 | 0 | 0 | 0 | 0 | 0 | 0 | 0 | 0 | 0 | 0 | 0 |
| MET B 463 | 0 | 0 | 0 | 0 | 0 | 0 | −13.307 | −3.330 | −10.472 | 0 | 0 | 0 |
| PRO B 464 | 0 | 0 | 0 | 0 | 0 | 0 | 0 | 0 | 0 | 0 | 0 | 0 |
| GLN B 465 | 0 | 0 | 0 | 0 | 0 | 0 | 0 | 0 | 0 | 0 | 0 | 0 |
| PRO B 466 | 0 | 0 | 0 | 0 | 0 | 0 | 0 | 0 | 0 | 0 | 0 | 0 |
| THR B 467 | 0 | 0 | 0 | 0 | 0 | 0 | 0 | 0 | 0 | 0 | 0 | 0 |
| PHE B 468 | 0 | 0 | 0 | 0 | 0 | 0 | 0 | −2.330 | 0 | 0 | 0 | 0 |
| THR B 469 | 0 | 0 | 0 | 0 | 0 | 0 | 0 | −3.330 | 0 | 0 | 0 | 0 |
| LEU B 470 | 0 | 0 | 0 | 0 | 0 | 0 | 0 | 0 | 0 | 0 | 0 | 0 |
| ARG B 471 | −20.098 | −14.530 | −3.808 | −8.593 | −23.171 | −7.233 | −23.307 | −2.330 | −11.472 | −12.976 | −16.528 | 0 |
| LYS B 472 | −26.098 | −21.530 | −4.808 | −11.593 | −26.171 | −16.233 | −21.307 | −3.330 | −14.472 | −13.976 | −20.528 | −15 |
| LYS B 473 | −21.098 | −13.530 | −3.808 | −20.593 | −13.171 | −14.233 | −14.307 | −2.330 | −12.472 | −8.976 | −14.528 | −15 |
| LEU B 474 | 0 | 0 | 0 | 0 | 0 | 0 | 0 | 0 | 0 | 0 | 0 | 0 |
| VAL B 475 | 0 | 0 | 0 | −10.593 | 0 | 0 | 0 | 0 | 0 | 0 | −19.528 | 0 |
| PHE B 476 | 0 | 0 | 0 | 0 | 0 | 0 | 0 | 0 | 0 | 0 | 0 | 0 |
| PRO B 477 | −22.098 | −12.530 | 0 | −10.593 | −16.171 | −7.233 | −22.307 | −2.330 | −9.472 | 0 | −30.528 | 0 |
| SER B 478 | 0 | 0 | 0 | 0 | 0 | 0 | 0 | 0 | 0 | 0 | 0 | 0 |
| ASP C B 479 | −22.098 | −10.530 | 0 | −13.593 | −15.171 | −15.233 | −25.307 | 0 | −9.472 | 0 | −30.528 | −14 |

While various embodiments of the present invention have been described above, it should be understood that they have been presented by way of example, and not limitation. It will be apparent to persons skilled in the relevant art that various changes in detail can be made therein without departing from the spirit and scope of the invention. Thus the present invention should not be limited by any of the above-described exemplary embodiments.

All references and publications referred to herein are hereby incorporated by reference in their entirety.

1. A method of determining the affinity between polypeptide amino acid residues and one or more molecular fragments comprising:
(a) conducting a computer simulation of (i) a polypeptide, and (ii) at least one molecular fragment, wherein at least one interaction energy is calculated between said polypeptide and said at least one molecular fragment, wherein said at least one calculated interaction energy is associated with a position of said at least one molecular fragment; and
(b) assigning an affinity value to at least one fragment and residue pair when said fragment is in the vicinity of the residue, wherein said affinity value is a measure of the free energy of interaction between the polypeptide and the fragment;
wherein (a) and (b) are conducted for each molecular fragment present in the computer simulation.

2. The method of claim 1, wherein said at least one fragment is defined as being in the vicinity of a residue when at least one pair of fragment-residue atoms is within a predetermined threshold distance, wherein said threshold distance is based on the sum of the Van der Waals radii of said fragment-residue atoms.

3. The method of claim 2, wherein said predetermined threshold distance is defined as:
rᵢⱼ<α(R_{VdW,i}+R_{VdW,j}),
wherein rᵢⱼ is the distance between the two atoms, R_{VdW} is the Van der Waals radius and α is a numerical parameter.

4. The method of claim 3, wherein said a is between about 0.5 and about 2.0.

5. The method of claim 4, wherein said a is about 1.2.

6. The method of claim 3, wherein said Van der Waals radius is about half the Lennard-Jones parameter from a molecular mechanics force-field.

7. The method of claim 6, wherein said molecular mechanics force field is selected from the group consisting of AMBER, GROMOS, CHARMM, Xplor, Discover, MMFFF and Tripos.

8. The method of claim 7, wherein said molecular mechanics force field is the AMBER force field.

9. The method of claim 8, wherein said affinity value comprises B-critical, wherein B critical is defined as the minimum B value for which a particular fragment is persistently observed in the vicinity of a residue, wherein B=μ′/kT+ln <N>, where μ′ is the excess chemical potential, k is the Boltzmann's constant, T is the absolute temperature, and <N> is the mean number of molecules of the molecular fragment.

10. The method of claim 9, wherein a particular type of fragment is persistently observed in the vicinity of a residue when the average number of fragments in the vicinity of the residue is between 0.8 and 1.0.

11. The method of claim 10, wherein a particular type of fragment is persistently observed in the vicinity of a residue when the average number of fragments in the vicinity is greater than or equal to 0.9.

12. The method of claim 8, wherein said affinity values comprise B-critical, wherein B critical is defined as
$${B}_{c} = - log [ \frac{1}{{n}_{snap}} {\sum }_{i = 1}^{{n}_{snap}} {\sum }_{frag j ∈ Δ {V}_{b}} {ⅇ}^{- {B}_{num} ( {Y}_{j} )} ] ;$$
wherein nₛₙₐₚ is a positive integer representing the number of snapshots from the numerical fragment density distribution, wherein Bₙᵤₘ(Yⱼ) is a field in the single particle configuration space Y, wherein said field represents an energy cost for a particular particle to leave the system from position Y; and )V_{b} is the binding volume.

13. The method of claim 1, further comprising outputting a binding analysis profile, wherein said binding analysis profile comprises a matrix of affinity values for each fragment-residue pair.

14. The method of claim 1, wherein (a) and (b) are repeated for a plurality of fragment types.

15. The method of claim 14, wherein a matrix of affinity values are averaged over fragments types, and the polypeptide surface is coded according to fragment binding affinity.

16. The method of claim 15, wherein residues with highest fragment binding affinity values are displayed with a different color from the residues with the lowest affinity value.
